(19) United States
(12) Patent Application Publication
(10) Pub. No.: US 2013/0066653 A1
(43) Pub. Date: Mar. 14, 2013

(54) Apparatus and method for linking a pacemaker or a defibrillator to an electronic healthcare record

(71) Applicants: Raymond Anthony Joao, Yonkers, NY (US); Michael Nicholas Mantzouris, Scarsdale, NY (US)

(72) Inventors: Raymond Anthony Joao, Yonkers, NY (US); Michael Nicholas Mantzouris, Scarsdale, NY (US)

(21) Appl. No.: 13/573,285

(22) Filed: Sep. 8, 2012

Related U.S. Application Data
(60) Provisional application No. 61/573,620, filed on Sep. 10, 2011.

(51) Int. Cl. A61N 1/39; G06Q 50/24; G06Q 40/08; A61N 1/362

(52) U.S. Cl. 705/3; 607/7; 607/30

(57) ABSTRACT

A computer-implemented method, including accessing, with a communication device, a computer in which is stored a healthcare record, or a medical record, of, for, or regarding, an individual or a patient, accessing, with the communication device, the healthcare record or the medical record of, for, or regarding, the individual or the patient, accessing or linking to a pacemaker, a pacemaker monitor, a defibrillator, or a defibrillator monitor, of or associated with the individual or the patient, with or using the communication device, and performing, via, with, or using, the communication device, a control, a monitoring, a diagnostic, a programming, a re-programming, a repair, a calibration, a setting, or a re-setting, action, operation, or function, on, regarding, or relating to, the pacemaker, the pacemaker monitor, the defibrillator, or the defibrillator monitor.

### RELATED APPLICATIONS

This application claims the benefit of priority of U.S. Provisional Patent Application Ser. No. 61/573,620, filed Sep. 10, 2011, and entitled “APPARATUS AND METHOD FOR LINKING A PACEMAKER OR A DEFIBRILLATOR TO AN ELECTRONIC HEALTHCARE RECORD”, the subject matter and teachings of which are hereby incorporated by reference herein.

### FIELD OF THE INVENTION

The present invention pertains to an apparatus and a method for linking a pacemaker or a defibrillator to an electronic healthcare record and, in particular, to an apparatus and a method for linking a pacemaker or a defibrillator to an electronic healthcare record or an electronic medical record of a patient which can facilitate enhanced patient care and monitoring and enhanced pacemaker or defibrillator monitoring, control, servicing, and/or maintenance.

### BACKGROUND OF THE PRESENT INVENTION

Millions of individuals and patients around the world rely on pacemakers and/or defibrillators in order to survive. These devices are typically sophisticated and are typically implanted in an individual or patient, or otherwise located in very close proximity to the individual or patient. These devices can also require periodic monitoring, programming, maintenance, and/or servicing, in order to ensure proper operation so as to ensure the health and well-being for those who need to utilize them. Despite the healthcare industry's giant strides in electronic health record technology and/or electronic medical record technology, none of these technologies address the needs of individuals and patients who utilize and rely on pacemakers, defibrillators, or devices designed and utilized to monitor same.

### SUMMARY OF THE INVENTION

The present invention overcomes the shortcomings of the prior art by providing an apparatus and a method for linking a pacemaker or a defibrillator to an electronic healthcare record and, in particular, to an apparatus and a method for linking a pacemaker or a defibrillator to an electronic healthcare record or an electronic medical record of a patient which can facilitate enhanced patient care and monitoring and enhanced pacemaker or defibrillator monitoring, control, servicing, and/or maintenance.

The present invention pertains to an apparatus and a method for linking a pacemaker or a defibrillator to an electronic healthcare record and, in particular, to an apparatus and a method for linking a pacemaker or a defibrillator to an electronic healthcare record or an electronic medical record of a patient which can facilitate enhanced patient care and monitoring and enhanced pacemaker or defibrillator device monitoring, control, servicing, and/or maintenance.

The apparatus of the present invention can include a central processing computer or central processing computer system which can be a network or server computer or any other computer or computer system. The central processing computer can provide control over the apparatus of the present invention and can perform any of the various processing services and/or functions described herein. The central processing computer may be a single computer or system of computers and/or may be include a plurality of computers or computer systems which are utilized in conjunction with one another. The central processing computer can provide services for any of the other computers and/or computer systems described herein as being associated with any of the individuals, patients, healthcare providers, insurers, payers, brokers, agents, and/or intermediaries, described herein.

The apparatus of the present invention also includes a healthcare provider communication device or computer which is associated with a healthcare provider such as a healthcare professional, a hospital, a clinic, and/or any other provider of services described herein. Any number or amount of healthcare provider communication devices or computers can be utilized in conjunction with a healthcare provider and/or group of providers. The healthcare provider computer can communicate with, and operate in conjunction with, the central processing computer and/or any of the other computers and/or computer systems associated with any of the other individuals and/or entities which utilize and/or operate in conjunction with the present invention.

The apparatus of the present invention can also include a healthcare payer communication device or computer which is associated with a healthcare payer such as a healthcare insurer, insurance company, health maintenance organization, a clinic, and/or any other payer of healthcare services and products described herein. Any number or amount of healthcare payer communication devices or computers can be utilized in conjunction with a healthcare payer and/or group of payers. The healthcare payer computer can communicate with, and operate in conjunction with, central processing computer and/or any of the other computers and/or computer systems associated with any of the other individuals and/or entities which utilize and/or operate in conjunction with the present invention.

The apparatus of the present invention can also include a patient or individual user communication device or computer which is associated with a healthcare patient such as a patient, user, or client who seeks or who is provided with healthcare and/or related services, products and/or related information. The patient communication device can also be utilized by any individual, party, or entity, who or which may merely utilize the present invention in order to obtain information of interest.

A patient communication device may also be located at public places or locations, such as at kiosks or other publicly available computer or communication devices. Any number or amount of patient computers can be utilized in conjunction with a patient and/or group of patients. The patient communication device can communicate with, and operate in conjunction with, the central processing computer and/or any of the other computers and/or computer systems associated with any of the other individuals and/or entities which utilize and/or operate in conjunction with the present invention.

The apparatus of the present invention can also include an intermediary communication device or computer which is associated with an intermediary, a broker, an agent, and/or any other individual and/or entity, that can utilize the present invention in order to act for and/or on behalf of any other individual, party, or entity, described herein. Any number or amount of intermediary computers can be utilized in conjunction with an intermediary and/or group of intermediaries. The intermediary computer can communicate with, and operate in conjunction with, the central processing computer and any of the other computers and/or computer systems associated with any of the other individuals and/or entities which utilize and/or operate in conjunction with the present invention.

The apparatus of the present invention also includes a pacemaker which is device which is implanted in, or implantable in, the body of the patient. The pacemaker can also be located external from the body of the patient, or can be attachable to the body of the patient, or can be wearable by the patient or can be integrated with or attached to the clothing of the patient.

The pacemaker can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

The pacemaker can also include any of the components of, and/or can perform any and/or all of the functions or functionality of, any of the many commercially available pacemakers in the marketplace as of the filing date of the present application. Further, the pacemaker can also include a transmitter(s) and a receiver(s), and can include a memory device or a database containing any of the data or information needed, required, or desired, for facilitating the functionality described herein as being capable of being provided by the apparatus of the present invention, as well as for facilitating any bi-directional communication between the pacemaker and any of the other computers, communication devices, or other devices described herein.

The pacemaker is associated with the patient communication device of the patient, or a relative of the patient, in or for which the pacemaker is implanted or externally used. The pacemaker can communicate with, and operate in conjunction with, the central processing computer and/or any of the other computers and/or computer systems associated with any of the other individuals and/or entities which utilize and/or operate in conjunction with the present invention. Any number of pacemakers can be utilized in connection with the apparatus of the present invention.

The apparatus of the present invention also includes a pacemaker monitor which can be used to monitor an operation of the pacemaker, control any operation of the pacemaker, calibrate the pacemaker, diagnose an malfunction or a state of disrepair of the pacemaker, re-calibrate an operation of the pacemaker, or re-program an operation of the pacemaker. The pacemaker monitor can receive signals, data, or information, from the pacemaker as well as transmit signals, data, or information, to the pacemaker.

The pacemaker monitor can communicate with the pacemaker using wireless communication technology or wirelessly. The pacemaker monitor can also be connected via a wired connection to the pacemaker.

The pacemaker monitor be any commercially available pacemaker monitor which is suitable for use in monitoring the pacemaker and can also include any additional transmitter(s), receiver(s), memory device or database, and/or hardware or software needed, required, or desired, for performing any and/or all of the functions and/or functionality described herein as being performed by the pacemaker monitor, the apparatus of the present invention, and/or any of its component parts, as well as for facilitating any bi-directional communication between the pacemaker monitor and any of the other computers, communication devices, or other devices described herein.

The pacemaker monitor can be located external from the body of the patient, or can be attachable to the body of the patient, or can be wearable by the patient or can be integrated with or attached to the clothing of the patient.

The pacemaker monitor can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

The pacemaker monitor, at the very least, can include any of the components of, and/or can perform any and/or all of the functions or functionality of, any of the many commercially available pacemaker monitors in the marketplace as of the filing date of the present application.

The pacemaker monitor is associated with the pacemaker and/or with the patient communication device of the patient, or a relative of the patient, in or for which the pacemaker is implanted or externally used. The pacemaker monitor can communicate with, and operate in conjunction with, the pacemaker, the patient communication device, the central processing computer, and/or any of the other computers and/or computer systems associated with any of the other individuals and/or entities which utilize and/or operate in conjunction with the present invention. Any number of pacemaker monitors can be utilized in connection with the apparatus of the present invention.

The apparatus of the present invention also includes a defibrillator which is device which can be implanted in, or can be implantable in, the body of the patient. The defibrillator can also be located external from the body of the patient, or can be attachable to the body of the patient, or can be wearable by the patient or can be integrated with or attached to the clothing of the patient.

The defibrillator can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

The defibrillator, at the very least, can also include any of the components of, and/or can perform any and/or all of the functions or functionality of, any of the many commercially available defibrillators in the marketplace as of the filing date of the present application. The defibrillator can also include a transmitter(s) and a receiver(s), and can include a memory device or a database containing any of the data or information needed, required, or desired, for facilitating the functionality described herein as being capable of being provided by the apparatus of the present invention, as well as for facilitating any bi-directional communication between the defibrillator and any of the other computers, communication devices, or other devices described herein.

The defibrillator is associated with the patient communication device of the patient, or a relative of the patient, in or for which the defibrillator is implanted or externally used. The defibrillator can communicate with, and operate in conjunction with, the central processing computer and/or any of the other computers and/or computer systems associated with any of the other individuals and/or entities which utilize and/or operate in conjunction with the present invention. Any number of defibrillators can be utilized in connection with the apparatus of the present invention.

The apparatus of the present invention can includes a defibrillator monitor which, in a preferred embodiment, can be used to monitor an operation of the defibrillator, control the operation of the defibrillator, calibrate the defibrillator, diagnose a malfunction or a state of disrepair of the defibrillator, re-calibrate an operation of the defibrillator, or re-program an operation of the defibrillator. The defibrillator monitor can receive signals, data, or information, from the defibrillator as well as transmit signals, data, or information, to the defibrillator.

The defibrillator monitor can communicate with the defibrillator using wireless communication technology or wirelessly. The defibrillator monitor can also be connected via a wired connection to the defibrillator.

The defibrillator monitor can be any commercially available defibrillator monitor which is suitable for use in monitoring the defibrillator and can also include any additional transmitter(s), receiver(s), memory device or database, and/or hardware or software needed, required, or desired, for performing any and/or all of the functions and/or functionality described herein as being performed by the defibrillator monitor, the apparatus of the present invention, and/or any of its component parts, as well as for facilitating any bi-directional communication between the defibrillator monitor and any of the other computers, communication devices, or other devices described herein.

The defibrillator monitor can also be located external from the body of the patient, or can be attachable to the body of the patient, or can be wearable by the patient or can be integrated with or attached to the clothing of the patient. The defibrillator monitor can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

The defibrillator monitor can also include any of the components of, and/or can perform any and/or all of the functions or functionality of, any of the many commercially available defibrillator monitors in the marketplace as of the filing date of the present application. The defibrillator monitor can also be associated with the defibrillator and/or with the patient communication device of the patient, or a relative of the patient, in or for which the defibrillator is implanted or externally used.

The defibrillator monitor can also communicate with, and operate in conjunction with, the defibrillator, the patient communication device, the central processing computer, and/or any of the other computers and/or computer systems associated with any of the other individuals and/or entities which utilize and/or operate in conjunction with the present invention. Any number of defibrillator monitors can be utilized in connection with the apparatus of the present invention.

In the preferred embodiment, any of the provider computer(s), the payer computer(s), the patient computer(s), the intermediary computer(s), the pacemaker(s), the pacemaker monitor(s), the defibrillator(s), or the defibrillator monitor(s), described herein, can be, or can be integrated with or into, any computer or communication device, including, but not limited to, a personal computer, a home computer, a server computer, a network computer, a hand-held computer, a palmtop computer, a laptop computer, a personal communication device, a cellular telephone, wireless telephone, a personal digital assistant, a telephone, a digital telephone, a television, an interactive television, a beeper, a pager, and/or a watch.

Each of the central processing computer(s), the provider computer(s), the payer computer(s), the patient computer(s), the intermediary computer(s), the pacemaker(s), the pacemaker monitor(s), the defibrillator(s), or the defibrillator monitor(s), described herein can transmit information to, as well as receive information from, any of the computers, or any of the pacemakers, pacemaker monitors, defibrillators, and/or defibrillator monitors, described herein. In this regard, each of the computers, or any of the pacemakers, pacemaker monitors, defibrillators, and/or defibrillator monitors, described herein, can communicate with, process information from, and/or share data and/or information with, each other and/or any other computer or computers, or any of the pacemakers, pacemaker monitors, defibrillators, and/or defibrillator monitors, described herein and/or utilized in conjunction with the present invention. In this manner, data and/or information transfer between any of the computers, or any of the pacemakers, pacemaker monitors, defibrillators, and/or defibrillator monitors, described herein, can communicate with any other computer or computers, or any of the pacemakers, pacemaker monitors, defibrillators, and/or defibrillator monitors, described herein, in a bi-directional manner.

The central processing computer(s), the provider computer(s), the payer computers(s), the patient computer(s), the intermediary computer(s), the pacemaker(s), the pacemaker monitor(s), the defibrillator(s), and/or the defibrillator monitor(s), can communicate with one another, and/or be linked to one another, over a communication network, a telecommunication network, a telephone network, a line-connected network, and/or a wireless communication network. Each of the computers, and each of the pacemaker(s), the pacemaker monitor(s), the defibrillator(s), and/or the defibrillator monitor(s), can be linked with any other computer or computers, or any respective pacemaker, pacemaker monitor, defibrillator, and/or defibrillator monitor, directly or indirectly directly or indirectly with one another so as to facilitate a direct or indirect bi-directional communication between and among said respective computers, or any pacemakers, pacemaker monitors, defibrillators, or defibrillator monitors.

The present invention can be utilized on, and/or over, the Internet and/or the World Wide Web. The present invention can also utilize wireless Internet and/or World Wide Web services, equipment and/or devices. The central processing computer(s) can have a web site or web sites associated therewith.

The present invention can be utilized in connection with the Internet and/or the World Wide Web and/or can also be utilized with any appropriate communication network or system including, but not limited to, a communication network or system, a telecommunication network or system, a telephone communication network or system, a cellular communication network or system, a wireless communication network or system, a line or wired communication network or system, a wireless Internet network or system, a wireless World Wide Web network or system, a digital communication network or system, a personal communication network or system, a personal communication services (PCS) network or system, a satellite communication network or system, a broad band communication network or system, a low earth orbiting (LEO) satellite network or system, a public switched telephone network or system, a telephone communication network or system, a radio communication network or system, and/or any other communication network or system, and/or any combination of the above communication networks or systems.

Each of the central processing computer(s), the provider computer(s), the payer computer(s), the patient computer(s), intermediary computer(s), the pacemaker(s), the pacemaker monitor(s), the defibrillator(s), and/or the defibrillator monitor(s), can transmit data and/or information using TCP/IP, as well as any other Internet and/or World Wide Web, and/or communication, protocols.

The present invention can be utilized in numerous preferred embodiments in order to provide a vast array of healthcare and healthcare-related services for any one or more of the various parties described herein relating to, or regarding the monitoring, controlling, servicing, and/or maintenance, of the pacemakers, pacemaker monitors, defibrillators, and/or defibrillator monitors, described herein.

The present invention can be utilized by any authorized provider of an individual or patient, payer of the individual or patient, intermediary, third party, caregiver, relative, any other authorized individual or entity, or the patient or individual himself or herself, in order to access or link to, or be connected to, or be brought into communication with, a respective pacemaker, pacemaker monitor, defibrillator, or defibrillator monitor, of or associated with an individual or patient, via the individual's or patient's healthcare record, file, or history, or medical record, file, or history, in order to allow the respective and authorized provider of the individual or patient, payer of the individual or patient, intermediary, third party, caregiver, relative, any other authorized individual or entity, or the patient or individual himself or herself, to control an operation of, to activate, to de-activate, to enable, or to disable, the respective pacemaker, pacemaker monitor, defibrillator, or defibrillator monitor, to monitor an operation of, to transmit a signal, data, or information, to or to obtain or receive a signal, data, or information, from the respective pacemaker, pacemaker monitor, defibrillator, or defibrillator monitor, and/or to program, re-program, calibrate, set or reset, and/or to perform a diagnostic function on or regarding, the respective pacemaker, pacemaker monitor, defibrillator, or defibrillator monitor.

Each of the pacemakers, the pacemaker monitors, the defibrillators, or the defibrillator monitors, can also be programmed to generate and/or to send notification messages or alerts regarding the occurrence(s) of certain events or happenings. In this regard, each of the pacemakers, the pacemaker monitors, the defibrillators, or the defibrillator monitors, can be quipped with the needed, required, or desired, hardware and/or software for detecting the occurrence of the events or happenings, for generating the notification messages or alerts, and for transmitting the notification messages or alerts to a respective computer or communication device of a respective the respective provider, payer, patient, intermediary, third party, caregiver, relative, any other authorized individual or entity, or the patient or individual himself or herself.

A respective pacemaker, pacemaker monitor, defibrillator, or defibrillator monitor, can also detect and report on any occurrence, event, or happening, which warrants providing a notification or an alert to any provider, payer, patient, intermediary, third party, caregiver, relative, any other authorized individual or entity, or the patient or individual himself or herself, or any other user of the present invention.

An occurrence, event, or happening, can, for example, be, but is not limited to, a detected malfunction, failure, or state of disrepair, of a respective pacemaker, pacemaker monitor, defibrillator, or defibrillator monitor, an individual or patient having received an electric shock, an individual or patient having received Tachy Arrhythmia Therapy such as Anti-Tachycardia Pacing, an increase in a pacing threshold of an individual or patient, an individual's or patient's pacing threshold having increased so much that the respective pacemaker, pacemaker monitor, defibrillator, or defibrillator monitor, is no longer capturing an individual's or patient's pacing impedance having increased or decreased by more than a recommended amount by a lead, an individual's or patient's sensing threshold having increased or decreased by more than a recommended amount or limit, an individual's or patient's sensing threshold having changed so much that the respective pacemaker, pacemaker monitor, defibrillator, or defibrillator monitor, is no longer sensing or operating correctly or with the required or desired degree or level of confidence, the individual or patient having experienced an arrhythmia which the respective pacemaker, pacemaker monitor, defibrillator, or defibrillator monitor, has detected and has declared to be an episode, that the individual's or patient's respective pacemaker, pacemaker monitor, defibrillator, or defibrillator monitor, has enacted a feature unique to that particular device, that the individual or patient has another device(s) that the respective pacemaker monitor or defibrillator monitor is monitoring and which has reached a certain threshold so as to declare an alert or an alert regarding the health and well-being of the individual or patient, that the individual or patient has had or has experienced a change in status for any of the symptoms that the respective pacemaker, pacemaker monitor, defibrillator, or defibrillator monitor is monitoring or being used for, that the individual or patient, or any other authorized person, who has access to one of the respective pacemaker monitors or defibrillator monitors, has declared to be an emergency thereby triggering an alerting of one or more, or all, of the parties involved in an individual's or patient's care, that the respective pacemaker, pacemaker monitor, defibrillator, or defibrillator monitor, has declared a malfunction or a state of disrepair, that a respective pacemaker monitor or a defibrillator monitor can no longer make contact with or communicate with a respective pacemaker or defibrillator thereby triggering a need to declare an emergency, and/or any other occurrence, event, or happening, which may warrant the providing of notice or an alert to any of the individuals or entities described herein as using the present invention.

The respective pacemaker, pacemaker monitor, defibrillator, or defibrillator monitor, can also communicate in bi-directional manner with any one or more of the respective databases of any of the respective central processing computer(s), provider computer(s), payer computer(s), patient computer(s), or intermediary computer(s), and/or with any of the healthcare records, files, or histories, or medical records, files, or histories store therein or associated therewith.

The respective pacemaker, pacemaker monitor, defibrillator, or defibrillator monitor, can also provide and transmit real-time electrogram or real-time EKG data and/or information, and/or any data and/or any data and/or information regarding any arrhythmia(s), feature activation(s), or respective pacemaker, pacemaker monitor, defibrillator, or defibrillator monitor, malfunction(s), failure(s), or state(s) of disrepair, to any one or more of the respective databases of any of the respective central processing computer(s), provider computer(s), payer computer(s), patient computer(s), or intermediary computer(s), for storage thereat or therein, and/or for storage in any of the healthcare records, files, or histories, or medical records, files, or histories, stored therein or associated therewith.

The apparatus of the present invention and/or any of the herein-described central processing computer(s), the provider computer(s), the payer computers(s), the patient computer(s), and the intermediary computer(s), or any one or more of the pacemakers, the pacemaker monitors, the defibrillators, or the defibrillator monitors, can also be programmed to perform troubleshooting, diagnostics, and repair, and/or reprogramming, of any of the herein-described pacemakers, pacemaker monitors, defibrillators, or defibrillator monitors.

The apparatus of the present invention and/or any of the herein-described central processing computer(s), the provider computer(s), the payer computers(s), the patient computer(s), and the intermediary computer(s), or any one or more of the pacemakers, pacemaker monitors, defibrillators, or defibrillator monitors, can also be utilized to update any setting(s) of any of the herein-described pacemakers or defibrillators in order to illicit a desired response in the individual or patient or from the individual or patient.

The apparatus of the present invention and/or any of the herein-described central processing computer(s), the provider computer(s), the payer computers(s), the patient computer(s), and the intermediary computer(s), or any one or more of the pacemakers, pacemaker monitors, defibrillators, or defibrillator monitors, can also be programmed to send an emergency telephone call to a 911 facility, to a police department, to afire department, to an emergency medical services department, or any other entity in order to report an emergency, a life-threatening condition, or any other emergency situation.

Any of the pacemaker monitors or defibrillator monitors can be provided as a wearable device, as a bedside device, as a stand alone device, as a portable device, as a device which can be located in, or integrated in or with a vehicle, such as a motor vehicle, an automobile, a mass transportation vehicle, a bus, a train, a subway, a boat or marine vessel or vehicle, an aircraft, an airplane, a jet, a helicopter, and/or an ambulance or emergency vehicle or any land, air, or sea, variety, kind, or type. The respective pacemaker monitor or defibrillator monitor can also be programmed to automatically detect the presence of a pacemaker or a defibrillator, its model, type, or kind, and can also be programmed to become automatically activated, or to switch on automatically, in order to monitor and/or service the pacemaker or the defibrillator.

Any of the pacemaker monitors or defibrillator monitors utilized in connection with the present invention can also be integrated with a respective pacemaker or defibrillator. A respective pacemaker monitor and/or defibrillator monitor can also be programmable in order to allow any person or caregiver to be able to provide care for an individual or patient. In this regard, any problems arising relating to a respective pacemaker or defibrillator can be dealt with more rapidly.

A respective pacemaker monitor or defibrillator monitor can also be a stand alone device which can be equipped and/or programmed so as to perform any and/or all functions of a programmer device and/or any of the herein-described functions or functionality described herein as being performed by the present invention, or any of the provider computer(s) or communication device(s), payer computers(s), patient computer(s) or communication device(s), or intermediary computer(s) described herein.

A respective pacemaker monitor or defibrillator monitor can also contain a database which can store any of the herein-described healthcare records, files, or histories, or medical records, files, of histories, of or for the individual or patient. A respective pacemaker monitor or defibrillator monitor can also be equipped and/or programmed to make 911 emergency calls, collect symptom data and/or information from the individual or patient, perform a systematic check of the respective pacemaker or defibrillator, and/or perform a systematic self-check or self-test.

A respective pacemaker monitor or defibrillator monitor can also be equipped and/or programmed to provide a voice synthesizer which can ask questions of the individual or patient, record the questions and answer to same, transmit the questions and answers, in dialogue form, to any one of more of the central processing computer(s), the provider computer(s) or communication device(s), the payer computers(s), the patient computer(s) or communication device(s), and the intermediary computer(s), for storage in the respective healthcare records, files, or histories, or medical records, files, of histories, of or for the individual or patient, and/or transmit the questions and answers, in dialogue form, in a 911 emergency call, or to a communication device in a call to a provider, a hospital, an emergency medical technician, or any other emergency worker or healthcare worker.

Any of the herein-described central processing computer(s), provider computer(s) or communication device(s), payer computers(s), patient computer(s) or communication device(s), and/or intermediary computer(s), or any one or more of the pacemakers, pacemaker monitors, defibrillators, or defibrillator monitors, can also be voice-activated and/or voice-controllable.

The patient computer or communication device can be any computer, personal computer, cellular telephone, a personal digital assistant, a smartphone, or a tablet, or any other devices described herein as being used as a patient computer or communication device. A respective patient computer or communication device can also collect, store, and utilize, voice commands. A respective patient computer or communication device can also be programmed to generate messages, e-mail messages, test messages, SMS messages, or any other message and can transmit same to any of the herein-described central processing computer(s), provider computer(s) or communication device(s), payer computers(s), patient computer(s) or communication device(s), and/or intermediary computer(s), for storage in a respective healthcare record, file, or history, or medical record, file, of history, of or for the individual or patient, and/or can also transmit same to any appropriate or respective computer or communication device used by or associated with a provider, a payer, a caregiver, a family member, a next of kin, or any other authorized or designated person or entity. Any message(s) generated by and transmitted by the patient computer or communication device can also be or can include a transcript of what the individual or patient says, a translation from one language to another language of what the individual or patient says, or can be or can include a message which is typed by the individual or patient or by another person.

The present invention can also provide for any scheduled, random, on-the-fly, one-time, or any other, software and/or firmware upgrades, changes, settings, or calibrations, to and for any of the herein-described provider computer(s) or communication device(s), payer computers(s), patient computer(s) or communication device(s), and/or intermediary computer(s), or to and for any one or more of the pacemakers, pacemaker monitors, defibrillators, or defibrillator monitors, with a record of each upgrade to each respective computer or device being stored in the central processing computer as well as in each respective computer or device.

Any and/or all of the hardware, software, and/or functionality described herein as being performed by any of the herein-described central processing computer(s), the provider computer(s) or communication device(s), the payer computers(s), the patient computer(s) or communication device(s), and the intermediary computer(s), or any one or more of the pacemakers, the pacemaker monitors, the defibrillators, or the defibrillator monitors, as well as any of the hardware, software, and/or functionality of any of the herein-described central processing computer(s), the provider computer(s) or communication device(s), the payer computers(s), the patient computer(s) or communication device(s), and the intermediary computer(s), or any one or more of the pacemaker monitors or the defibrillator monitors, can be integrated designed into, built into, or integrated with any cellular telephone, smartphone, personal digital assistant, personal computer, laptop computer, notebook computer, tablet, or any other personal electronic device, used by or associated with any of the herein-described providers, payers, intermediaries, third parties, caregivers, relatives, any other authorized individuals or entities, or any individuals or patients themselves.

Any patient computer or patient communication device described herein as being utilized in connection with the present invention can serve as a respective pacemaker monitor, defibrillator monitor, or programmer device, and can be utilized to perform any of the functions described herein as being performed by a respective pacemaker monitor, defibrillator monitor, or programmer device, or perform any other functions which can be performed by a respective pacemaker monitor, defibrillator monitor, or programmer device.

The apparatus of the present invention can be utilized on or over, or in connection with, the Internet and/or the World Wide Web and/or on or over, or in connection with, any wireless communication networks or systems.

The present invention can also be utilized to allow any provider, payer, intermediary, or the individual or patient, or any other person, to access and/or link via a healthcare record, file, or history, or a medical record, file, or history, to a pacemaker, pacemaker monitor, defibrillator, or defibrillator monitor, and to perform any of the herein-described, or any other action, function, and operation. The present invention can also be utilized to allow a respective provider, intermediary, caregiver, or any other person or entity, to submit a claim for payment to an insurer or payer for any action, function, operation, or service, performed.

The present invention can also be utilized by any provider, payer, intermediary, or any individual or patient, or any other person, to perform spot checks or periodic checks on the operation of any of the herein-described pacemakers, pacemaker monitors, defibrillators, or defibrillator monitors.

The present invention can also be utilized to record and store any and/or all of the activities, events, occurrences, or happenings, concerning or regarding a respective pacemaker, pacemaker monitor, defibrillator, or defibrillator monitor, or any individual or patient using same.

Applicant further incorporates by reference herein, as if to fully restate herein, in its entirety or in summary form and as additional information regarding the summary of the present invention, the subject matter and teachings provided herein in the Description Of The Preferred Embodiments section of this application.

### BRIEF DESCRIPTION OF THE DRAWINGS

In the Drawings:

FIG. 1 illustrates a preferred embodiment of the present invention, in block diagram form;

FIG. 2 illustrates the central processing computer of FIG. 1, in block diagram form;

FIG. 3 illustrates the provider communication device of FIG. 1, in block diagram form;

FIG. 4 illustrates the payer communication of FIG. 1, in block diagram form;

FIG. 5 illustrates the patient communication device of FIG. 1, in block diagram form;

FIG. 6 illustrates the intermediary communication device of FIG. 1, in block diagram form;

FIGS. 7A and 7B illustrate a preferred embodiment method of using the present invention, in flow diagram form; and

FIG. 8 illustrates another preferred embodiment method of using the present invention, in flow diagram form.

### DESCRIPTION OF THE PREFERRED EMBODIMENTS

The present invention pertains to an apparatus and a method for linking a pacemaker or a defibrillator to an electronic healthcare record and, in particular, to an apparatus and a method for linking a pacemaker or a defibrillator to an electronic healthcare record or an electronic medical record of a patient which can facilitate enhanced patient care and monitoring and enhanced pacemaker or defibrillator device monitoring, control, servicing, and/or maintenance.

Applicant hereby incorporates by reference herein the subject matter and teachings of U.S. Provisional Patent Application Ser. No. 61/573,620, filed Sep. 10, 2011, and entitled “APPARATUS AND METHOD FOR LINKING A PACEMAKER OR A DEFIBRILLATOR TO AN ELECTRONIC HEALTHCARE RECORD”.

As used herein, the terms “individual”, “patient”, “client”, “user” or the like, or their plural forms, refers to any person, individual, patient, and/or client who uses the present invention, and/or who seeks and/or who receives healthcare services, healthcare-related services, healthcare-related information, and/or any of the other services and/or products provided by the present invention.

As used herein, the terms “doctor”, “healthcare provider”, “provider”, “therapist”, “healthcare information specialist”, etc., or their plural forms, refers to any medical doctor, including any and all of the various medical specialists and/or specialties, including, but not limited to internists, orthopedists, opthamalogists, cardiologists, hematologists, endocrinologists, oncologists, ears, nose and throat specialists, neurologists, urologists, gastrointerologists, dermatologists, pediatricians), medical specialist, surgeon, surgical specialists, including any and/or forms and/or types of surgeons), physician, dentist, psychiatrist, psychologist, optometrist, podiatrist, osteopath, chiropractor, pharmacist, therapist, physical therapist, respiratory therapist, nurse, healthcare aid, nutritionist, and/or any other person, individual and/or professional who can provide healthcare, healthcare-relate, wellness and/or wellness-related services and/or products.

As used herein, the terms “insurer”, “payer”, “insurance provider”, “heath insurance provider”, “life insurance provider”, “disability insurance provider”, etc., or their plural forms, refers to any insurance companies, healthcare insurance companies, disability insurance companies, casualty insurance companies, health maintenance organizations, healthcare providers, and any other payer and/or provider of healthcare services and/or products, who which provide and/or pay for healthcare and/or healthcare-related benefits, services, and/or products, and/or who or which provide respective health insurance, life insurance and/or disability insurance benefits, services and/or products.

As used herein, the terms “intermediary”, “broker”, “agent”, “billing service”, “collection agent”, “manager”, “assistant”, etc., or their plural forms, refer to any intermediary, third party, pacemaker manufacturer, pacemaker provider, pacemaker servicing individual or entity, pacemaker vendor, pacemaker servicing representative, individual, or entity, pacemaker monitoring representative, individual, or entity, defibrillator manufacturer, defibrillator provider, defibrillator servicing individual or entity, defibrillator vendor, defibrillator servicing representative, individual, or entity, defibrillator monitoring representative, individual, or entity, emergency medical services provider, individual, entity, or agency, broker, insurance broker, agent, insurance agent, billing service provider, collection agent, claim processing agent, and/or any other person, individual, and/or entity, which acts on behalf of, or for, any of the individuals, patients, doctors, healthcare providers, insurers, payers, etc., described herein.

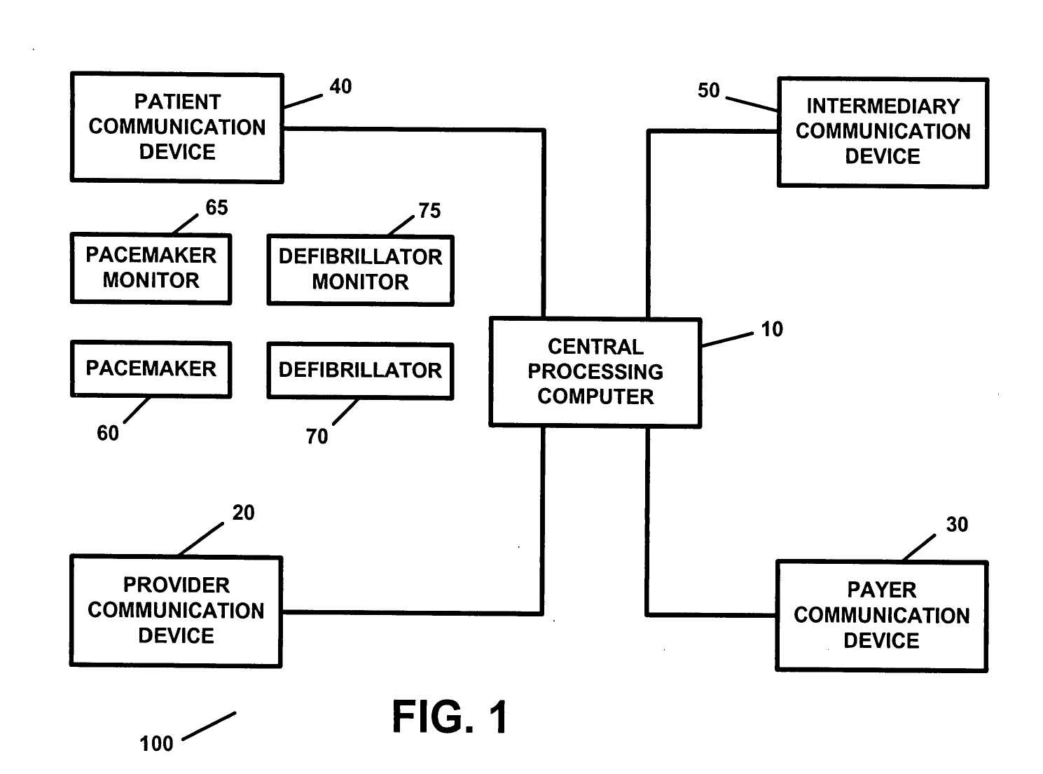

FIG. 1 illustrates a preferred embodiment of the apparatus of the present invention, designated by the reference numeral 100, in block diagram form. With reference to FIG. 1, the apparatus 100 includes a central processing computer or central processing computer system 10 (hereinafter referred to as the “central processing computer 10”). In the preferred embodiment the central processing computer 10 can be a network or server computer.

In the preferred embodiment, the central processing computer 10 can provide control over the apparatus 100 and can perform any of the various processing services and/or functions described herein. The central processing computer 10 may be a single computer or system of computers and/or may be include a plurality of computers or computer systems which are utilized in conjunction with one another. The central processing computer 10, in the preferred embodiment can provides services for any of the other computers and/or computer systems described herein as being associated with any of the individuals, patients, healthcare providers, insurers, payers, brokers, agents, and/or intermediaries, described herein.

The apparatus 100 also includes a healthcare provider communication device or computer 20 (hereinafter referred to as “provider communication device 20” or “provider computer 20”) which is associated with a healthcare provider such as a healthcare professional, a hospital, a clinic, and/or any other provider of services described herein. Any number or amount of healthcare provider computers 20 can be utilized in conjunction with a healthcare provider and/or group of providers. The healthcare provider computer(s) 20 can communicate with, and operate in conjunction with, the central processing computer 10 and/or any of the other computers and/or computer systems associated with any of the other individuals and/or entities which utilize and/or operate in conjunction with the present invention.

The apparatus 100 can also include a healthcare payer communication device or computer 30 (hereinafter referred to as “payer communication device 30” or “payer computer 30”) which is associated with a healthcare payer such as a healthcare insurer, insurance company, health maintenance organization, a clinic, and/or any other payer of healthcare services and products described herein. Any number or amount of healthcare payer computers 30 can be utilized in conjunction with a healthcare payer and/or group of payers. The healthcare payer computer(s) 30 can communicate with, and operate in conjunction with, central processing computer 10 and/or any of the other computers and/or computer systems associated with any of the other individuals and/or entities which utilize and/or operate in conjunction with the present invention.

The apparatus 100 can also include a patient or individual user communication device or computer 40 (hereinafter “patient communication device 40” or “patient computer 40”) which is associated with a healthcare patient such as a patient, user, or client who seeks or who is provided with healthcare and/or related services, products and/or related information. The patient communication device 40 can also be utilized by any individual, party, or entity, who or which may merely utilize the present invention in order to obtain information of interest.

A patient communication device 40 may also be located at public places or locations, such as at kiosks or other publicly available computer or communication devices. Any number or amount of patient computers 40 can be utilized in conjunction with a patient and/or group of patients. The patient communication device 40 can communicate with, and operate in conjunction with, the central processing computer 10 and/or any of the other computers and/or computer systems associated with any of the other individuals and/or entities which utilize and/or operate in conjunction with the present invention.

The apparatus 100 can also include an intermediary communication device or computer 50 (hereinafter referred to as “intermediary communication device 50” or “intermediary computer 50”) which is associated with an intermediary, a broker, an agent, and/or any other individual and/or entity, that can utilize the present invention in order to act for and/or on behalf of any other individual, party, or entity, described herein. Any number or amount of intermediary computers 50 can be utilized in conjunction with an intermediary and/or group of intermediaries. The intermediary computer(s) 50 can communicate with, and operate in conjunction with, the central processing computer 10 and any of the other computers and/or computer systems associated with any of the other individuals and/or entities which utilize and/or operate in conjunction with the present invention.

The apparatus 100 also includes a pacemaker 60 which is device which, in a preferred embodiment, is implanted in, or implantable in, the body of the patient. In another preferred embodiment, the pacemaker 60 can also be located external from the body of the patient, or can be attachable to the body of the patient, or can be wearable by the patient or can be integrated with or attached to the clothing of the patient.

In another preferred embodiment, the pacemaker 60 can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

In the preferred embodiment, the pacemaker 60, at the very least, can include any of the components of, and/or can perform any and/or all of the functions or functionality of, any of the many commercially available pacemakers in the marketplace as of the filing date of the present application. Further, in the preferred embodiment, the pacemaker 60 can also include a transmitter(s) and a receiver(s), and can include a memory device or a database containing any of the data or information needed, required, or desired, for facilitating the functionality described herein as being capable of being provided by the apparatus 100 of the present invention, as well as for facilitating any bi-directional communication between the pacemaker 60 and any of the other computers, communication devices, or other devices described herein.

In the preferred embodiment, the pacemaker 60 is associated with the patient communication device 40 of the patient, or a relative of the patient, in or for which the pacemaker is implanted or externally used.

The pacemaker 60 can communicate with, and operate in conjunction with, the central processing computer 10 and/or any of the other computers and/or computer systems associated with any of the other individuals and/or entities which utilize and/or operate in conjunction with the present invention.

Any number of pacemakers 60 can be utilized in connection with the apparatus 100 of the present invention.

The apparatus 100 also includes a pacemaker monitor 65 which, in a preferred embodiment, can be used to monitor an operation of the pacemaker 60, control any operation of the pacemaker 60, calibrate the pacemaker 60, diagnose an malfunction or a state of disrepair of the pacemaker 60, re-calibrate an operation of the pacemaker 60, or re-program an operation of the pacemaker 60. In a preferred embodiment, the pacemaker monitor 65 can receive signals, data, or information, from the pacemaker 60 as well as transmit signals, data, or information, to the pacemaker 60.

In a preferred embodiment, the pacemaker monitor 65 can communicate with the pacemaker 60 using wireless communication technology or wirelessly. In another preferred embodiment, in certain instances, the pacemaker monitor 65 can also be connected via a wired connection to the pacemaker 60.

In the preferred embodiment, the pacemaker monitor 65 be any commercially available pacemaker monitor 65 which is suitable for use in monitoring the pacemaker 60 and can also include any additional transmitter(s), receiver(s), memory device or database, and/or hardware or software needed, required, or desired, for performing any and/or all of the functions and/or functionality described herein as being performed by the pacemaker monitor 65, the apparatus 100 of the present invention, and/or any of its component parts, as well as for facilitating any bi-directional communication between the pacemaker monitor 65 and any of the other computers, communication devices, or other devices described herein.

In another preferred embodiment, the pacemaker monitor 65 can be located external from the body of the patient, or can be attachable to the body of the patient, or can be wearable by the patient or can be integrated with or attached to the clothing of the patient.

In another preferred embodiment, the pacemaker monitor 65 can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

In the preferred embodiment, the pacemaker monitor 65, at the very least, can include any of the components of, and/or can perform any and/or all of the functions or functionality of, any of the many commercially available pacemaker monitors in the marketplace as of the filing date of the present application.

In the preferred embodiment, the pacemaker monitor 65 is associated with the pacemaker 60 and/or with the patient communication device 40 of the patient, or a relative of the patient, in or for which the pacemaker is implanted or externally used.

The pacemaker monitor 65 can communicate with, and operate in conjunction with, the pacemaker 60, the patient communication device 40, the central processing computer 10, and/or any of the other computers and/or computer systems associated with any of the other individuals and/or entities which utilize and/or operate in conjunction with the present invention.

Any number of pacemaker monitors 65 can be utilized in connection with the apparatus 100 of the present invention.

The apparatus 100 also includes a defibrillator 70 which is device which, in a preferred embodiment, is implanted in, or implantable in, the body of the patient. In another preferred embodiment, the defibrillator 70 can also be located external from the body of the patient, or can be attachable to the body of the patient, or can be wearable by the patient or can be integrated with or attached to the clothing of the patient.

In another preferred embodiment, the defibrillator 70 can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

In the preferred embodiment, the defibrillator 70, at the very least, can include any of the components of, and/or can perform any and/or all of the functions or functionality of, any of the many commercially available defibrillators in the marketplace as of the filing date of the present application. Further, in the preferred embodiment, the defibrillator 70 can also include a transmitter(s) and a receiver(s), and can include a memory device or a database containing any of the data or information needed, required, or desired, for facilitating the functionality described herein as being capable of being provided by the apparatus 100 of the present invention, as well as for facilitating any bi-directional communication between the defibrillator 70 and any of the other computers, communication devices, or other devices described herein.

In the preferred embodiment, the defibrillator 70 is associated with the patient communication device 40 of the patient, or a relative of the patient, in or for which the defibrillator 70 is implanted or externally used.

The defibrillator 70 can communicate with, and operate in conjunction with, the central processing computer 10 and/or any of the other computers and/or computer systems associated with any of the other individuals and/or entities which utilize and/or operate in conjunction with the present invention.

Any number of defibrillators 70 can be utilized in connection with the apparatus 100 of the present invention.

The apparatus 100 also includes a defibrillator monitor 75 which, in a preferred embodiment, can be used to monitor an operation of the defibrillator 70, control the operation of the defibrillator 70, calibrate the defibrillator 70, diagnose a malfunction or a state of disrepair of the defibrillator 70, re-calibrate an operation of the defibrillator 70, or re-program an operation of the defibrillator 70. In a preferred embodiment, the defibrillator monitor 75 can receive signals, data, or information, from the defibrillator 70 as well as transmit signals, data, or information, to the defibrillator 70.

In a preferred embodiment, the defibrillator monitor 75 can communicate with the defibrillator 70 using wireless communication technology or wirelessly. In another preferred embodiment, in certain instances, the defibrillator monitor 75 can also be connected via a wired connection to the defibrillator 70.

In the preferred embodiment, the defibrillator monitor 75 be any commercially available defibrillator monitor 75 which is suitable for use in monitoring the defibrillator 70 and can also include any additional transmitter(s), receiver(s), memory device or database, and/or hardware or software needed, required, or desired, for performing any and/or all of the functions and/or functionality described herein as being performed by the defibrillator monitor 75, the apparatus 100 of the present invention, and/or any of its component parts, as well as for facilitating any bi-directional communication between the defibrillator monitor 75 and any of the other computers, communication devices, or other devices described herein.

In another preferred embodiment, the defibrillator monitor 75 can be located external from the body of the patient, or can be attachable to the body of the patient, or can be wearable by the patient or can be integrated with or attached to the clothing of the patient.

In another preferred embodiment, the defibrillator monitor 75 can also be located at, in, or associated with, any premises, a residential premises, a home of the patient, a provider office, a hospital, a provider facility, a vehicle, an aircraft or helicopter, a boat or marine vehicle or vessel, a recreational vehicle, an ambulance, an emergency vehicle, a police vehicle, a fire vehicle, or any other premises or vehicle on or in which the patient may be located, cared for, traveling in, or being transported in.

In the preferred embodiment, the defibrillator monitor 75, at the very least, can include any of the components of, and/or can perform any and/or all of the functions or functionality of, any of the many commercially available defibrillator monitors in the marketplace as of the filing date of the present application.

In the preferred embodiment, the defibrillator monitor 75 is associated with the defibrillator 70 and/or with the patient communication device 40 of the patient, or a relative of the patient, in or for which the defibrillator is implanted or externally used.

The defibrillator monitor 75 can communicate with, and operate in conjunction with, the defibrillator 70, the patient communication device 40, the central processing computer 10, and/or any of the other computers and/or computer systems associated with any of the other individuals and/or entities which utilize and/or operate in conjunction with the present invention.

Any number of defibrillator monitors 75 can be utilized in connection with the apparatus 100 of the present invention.

In the preferred embodiment, any of the provider computer(s) 20, the payer computer(s) 30, the patient computer(s) 40, the intermediary computer(s) 50, the pacemaker(s) 60, the pacemaker monitor(s) 65, the defibrillator(s) 70, or the defibrillator monitor(s) 75, described herein, can be, or can be integrated with or into, any computer or communication device, including, but not limited to, a personal computer, a home computer, a server computer, a network computer, a hand-held computer, a palmtop computer, a laptop computer, a personal communication device, a cellular telephone, wireless telephone, a personal digital assistant, a telephone, a digital telephone, a television, an interactive television, a beeper, a pager, and/or a watch.

Each of the central processing computer(s) 10, the provider computer(s) 20, the payer computer(s) 30, the patient computer(s) 40, the intermediary computer(s) 50, the pacemaker(s) 60, the pacemaker monitor(s) 65, the defibrillator(s) 70, or the defibrillator monitor(s) 75, described herein can transmit information to, as well as receive information from, any of the computers 10, 20, 30, 40, and/or 50, or any of the pacemakers 60, pacemaker monitors 65, defibrillators 70, and/or defibrillator monitors 75, described herein. In this regard, each of the computers 10, 20, 30, 40, and/or 50, or any of the pacemakers 60, pacemaker monitors 65, defibrillators 70, and/or defibrillator monitors 75, described herein, can communicate with, process information from, and/or share data and/or information with, each other and/or any other computer or computers 10, 20, 30, 40, and/or 50, or any of the pacemakers 60, pacemaker monitors 65, defibrillators 70, and/or defibrillator monitors 75, described herein and/or utilized in conjunction with the present invention. In this manner, data and/or information transfer between any of the computers 10, 20, 30, 40, and/or 50, or any of the pacemakers 60, pacemaker monitors 65, defibrillators 70, and/or defibrillator monitors 75, described herein, can communicate with any other computer or computers 10, 20, 30, 40, and/or 50, or any of the pacemakers 60, pacemaker monitors 65, defibrillators 70, and/or defibrillator monitors 75, described herein, in a bi-directional manner.

The central processing computer(s) 10, the provider computer(s) 20, the payer computers(s) 30, the patient computer(s) 40, the intermediary computer(s) 50, the pacemaker(s) 60, the pacemaker monitor(s) 65, the defibrillator(s) 70, and/or the defibrillator monitor(s) 75, can communicate with one another, and/or be linked to one another, over a communication network, a telecommunication network, a telephone network, a line-connected network, and/or a wireless communication network. Each of the computers 10, 20, 30, 40, and 50, and each of the pacemaker(s) 60, the pacemaker monitor(s) 65, the defibrillator(s) 70, and/or the defibrillator monitor(s) 75, can be linked with any other computer or computers, or any respective pacemaker 60, pacemaker monitor 65, defibrillator 70, and/or defibrillator monitor 75, directly or indirectly directly or indirectly with one another so as to facilitate a direct or indirect bi-directional communication between and among said respective computers, or any pacemakers 60, pacemaker monitors 65, defibrillators 70, or defibrillator monitors 75.

In the preferred embodiment, the present invention is utilized on, and/or over, the Internet and/or the World Wide Web. The present invention, in the preferred embodiment, can also utilize wireless Internet and/or World Wide Web services, equipment and/or devices. The central processing computer(s) 10, in the preferred embodiment, has a web site or web sites associated therewith.

Although the Internet and/or the World Wide Web is a preferred communication system and/or medium utilized, the present invention, in all of the embodiments described herein, can also be utilized with any appropriate communication network or system including, but not limited to, a communication network or system, a telecommunication network or system, a telephone communication network or system, a cellular communication network or system, a wireless communication network or system, a line or wired communication network or system, a wireless Internet network or system, a wireless World Wide Web network or system, a digital communication network or system, a personal communication network or system, a personal communication services (PCS) network or system, a satellite communication network or system, a broad band communication network or system, a low earth orbiting (LEO) satellite network or system, a public switched telephone network or system, a telephone communication network or system, a radio communication network or system, and/or any other communication network or system, and/or any combination of the above communication networks or systems.

In the preferred embodiment, each of the central processing computer(s) 10, the provider computer(s) 20, the payer computer(s) 30, the patient computer(s) 40, intermediary computer(s), the pacemaker(s) 60, the pacemaker monitor(s) 65, the defibrillator(s) 70, and/or the defibrillator monitor(s) 75, can transmit data and/or information using TCP/IP, as well as any other Internet and/or World Wide Web, and/or communication, protocols.

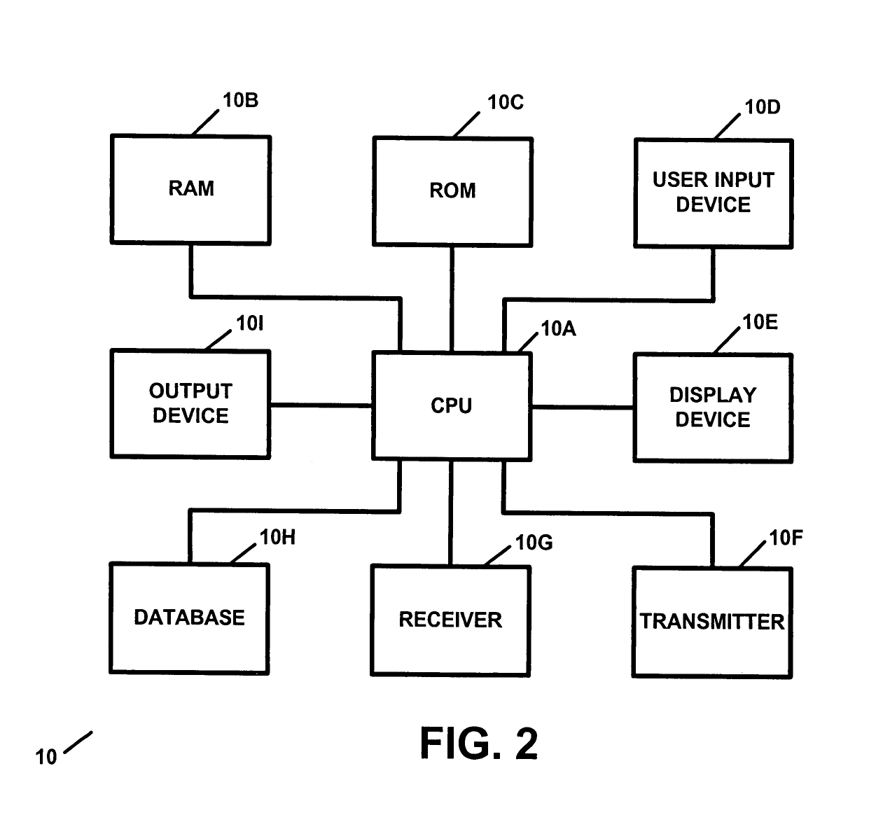

FIG. 2 illustrates the central processing computer 10, in block diagram form. The central processing computer 10, in the preferred embodiment, is a network computer or computer system, or any other communication device which can provide the functionality of, and which can be utilized as a central processing computer such as an Internet server computer and/or a web site server computer. In the preferred embodiment, the central processing computer 10 includes a central processing unit or CPU 10A, which in the preferred embodiment, is a microprocessor. The CPU 10A may also be a microcomputer, a minicomputer, a macro-computer, and/or a mainframe computer, depending upon the application.

The central processing computer 10 also includes a random access memory device(s) 10B (RAM) and a read only memory device(s) 10C (ROM), each of which is connected to the CPU 10A, a user input device 10D, for entering data and/or commands into the central processing computer 10, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electrocardiograms, blood-sugars monitors, etc., or any of the herein-described pacemakers 60, pacemaker monitors 65, defibrillators 70, or defibrillator monitors 75, if desired, which input device(s) are can also connected to or otherwise linked to the CPU 10A, either directly or indirectly via any of the herein-described computers 10, 20, 30, 40, and 50. The central processing computer 10 also includes a display device 10E for displaying data and/or information to a user or operator.

The central processing computer 10 also includes a transmitter(s) 10F, for transmitting signals and/or data and/or information to any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention. The central processing computer 10 also includes a receiver 10G, for receiving signals and/or data and/or information from any one or more of the central processing computer(s) 10, the provider computer(s) 20, the payer computers(s) 30, the patient computer(s) 40, and the intermediary computer(s) 50, or from any one or more of the pacemakers 60, the pacemaker monitors 65, the defibrillators 70, or the defibrillator monitors 75, which may be utilized in conjunction with the present invention.

The central processing computer 10 also includes a database(s) 10H which contains data and/or information pertaining to the patients, providers, payers, and intermediaries who or which are serviced by the present invention and/or who or which utilize the present invention. For each patient using a pacemaker or a defibrillator, information regarding the pacemaker or the defibrillator used in, implanted in, or utilized by, each patient can also be stored in the database(s) 10H.

The database 10H can contain any and/or all of the information needed and/or required in order to perform any and/or all of the functions, services and/or operations described herein as being performed by the central processing computer 10 or the apparatus 100 of the present invention. In this regard the database 10H can contain data and/or information regarding patient name, patient identification information, patient social security number or other identification information, date of birth, doctors or providers, therapists, nutritionists, insurance or payer information, group insurance information, group health insurance information, life insurance information, disability insurance information, patient address, phone number, e-mail and/or other contact information, medical history, psychological history, dental history, family history, family medical, psychological, and/or dental history, insurance coverage, insurance co-payment and/or deductible information, insurance information, insurance claim procedures, insurance claim forms, doctor or provider appointment schedules, past treatments, past diagnosis, symptoms, insurance claim forms, employer information, lifestyle information, treatment plans, treatment progress, broker/agent/intermediary information, education information, age, sex, marital status, employee benefits information, types or services and/or treatments needed, and any other data and/or information regarding the patient which would be needed and/or desired in order to perform any and/or all of the functions, services and/or operations described herein.

In cases where an individual or patient has prescribed or otherwise provided with, fitted with, or implanted with, or otherwise assigned with, a pacemaker or a defibrillator, or both, the database 10H can also contain data and/or information regarding the manufacturer and model of the respective pacemaker, pacemaker monitor, defibrillator, or defibrillator monitor, the prescribing provider or providers, the implanting or fitting provider or providers, date(s) of prescription, time(s) and/or date(s) of implanting, time(s) and/or date(s) of fitting, time(s) and/or date(s) put into service for the individual or patient, and/or any other data or information pertinent to the individual's or the patient's respective pacemaker, pacemaker monitor, defibrillator, and/or defibrillator monitor.

The database 10H can also contain information for accessing, a link, or a hyperlink, allowing any of the herein-described providers, payers intermediaries, individuals, patients, or any other authorized user to access the respective pacemaker 60, pacemaker monitor 65, defibrillator 70, or defibrillator monitor 75, of or for the individual or patient.

The database 10H can also contain information for accessing or obtaining, a link, or a hyperlink, to data or information regarding specifications, product descriptions, diagnostic information, serving information, maintenance information, calibration or re-calibration information, programming or re-programming or repair information, or any instructions or procedures regarding or relating to same.

The database(s) 10H can also contain healthcare and/or medical video, image, and/or audio, data and/or information, such as, for example, x-rays, Magnetic Resonant Images (MRI), CAT scans, digital x-ray files, digital Magnetic Resonant Imaging (MRI) files, digital CAT scan files, and/or any other video, imaging, and/or audio, healthcare data and/or information which can be utilized by healthcare providers, payers, intermediaries, patients, and/or other users of the present invention. In this manner, the present invention can facilitate the availability of any of the above-described video, image, and/or audio, data and/or information in a network environment. For example, a medical specialist can have access to, and/or review, an MRI or a CAT scan for a patient, from any location and at any time. A medical specialist can also access information regarding the operation of, or past operation of, any pacemaker or any defibrillator being utilized in connection with any patient.

The database 10H can also contain data and/or information regarding providers including provider name, provider social security number or identification number, type of professional or service provider, address, phone number, fax number, e-mail and/or other contact information, experience, specialties, insurances accepted, schedule of charges, financial account identification information, resume information, education, work experience, claim forms, appointment schedules, procedures performed, and/or any other data and/or information concerning the providers for providing any and/or all of the functions, services, and/or operations described herein as being performed by the present invention.

The database 10H can also contain data and/or information regarding all possible fields of medicine, surgery, psychiatry, psychology, dentistry, oral surgery, optometry, podiatry, physical therapy, respiratory therapy, hypnosis, osteopathy, nutrition, wellness, and/or any other possible healthcare fields and/or subject matter which can possibly by utilized in the processing and/or operation of the present invention.

The database 10H can also contain information on illnesses, symptoms, diseases and/or sicknesses, theories, scientific theories, research data and/or information, diagnosis information, treatment information, treatment plans, treatment processes, treatment progresses, treatment interactions, side effects, expected treatment results, treatment providers, treatment durations, treatment costs, pre-treatment information, post-treatment information, treatment monitoring information, statistical information regarding diagnoses, treatments, treatment success rates, treatment failure rates, treatment centers, therapy plans, therapy success rates, therapy failure rates, treatment procedures, medications treatments, non-medication treatments, healthcare institutions, treatment evaluating criteria, treatment mistakes and/or mishaps, indicators of mistakes and/or mishaps, corrective actions, links to providers, links to treatment centers or institutions, reimbursement rates, nutrition information, diet information, exercise information, exercise routines, treatment options, healthcare advise, wellness advice, preventive care, preventive procedure, health maintenance, drug and medication information, drug interaction information, video information, including video files or clips and other information, regarding illnesses, diseases, treatments and follow-up care, audio information, including audio files or clips and other information, regarding illnesses, diseases, treatments and follow-up care, treatment and/or procedure information and/or narratives, treatment analysis, diagnosis analysis, diagnosis monitoring, diagnosis confirmation and/or checking, and/or other information for providing the herein-described functions, services, and/or operations.

The database 10H can also contain information regarding the insurance companies and payers described herein, including, but not limited to, payer name, address, phone number, fax number, e-mail address, identification number(s), coverage types, policies and/or coverages provided, reimbursement rates, patients and/or providers serviced and/or covered by the payer, policy information, claim forms, claim procedures, claim status, claim processing information, claim submission procedures and policies, reasonable and customary charges, co-payment information, pre-approval information and/or procedures, claim form information, electronic form claim forms, insurance and/or coverage requirements, guidelines, and/or triggering events, covered procedures and/or treatments, uncovered procedures and/or treatments, claim approval information, claim approval history, claim approval statistics, claim rejection or denial information, claim rejection or denial history, claim rejection or denial statistics, financial account information, network provider information, network patient information, claim statistics, preventative care and/or benefits information, benefits information, benefits request information and/or claim forms, claim submission information, claim processing information, claim status information, payment information and statistics, and/or any other data and/or information regarding and/or related to payers which are needed and/or desired for providing any and/or all of the functions, services, and/or operations described herein.

The database 10H can also contain data and/or information regarding the brokers, agents and/or intermediaries described herein, including, but not limited to, intermediary name, address, phone number, fax number, e-mail address, clients, patients services, insurance policies, policy information, policy quote information, policy proposal information, any and/or all of the above information described herein regarding patients, providers, payers, etc. which may be of interest to the intermediaries described herein which may be useful and/or beneficial to the intermediaries in providing any of the functions, services, and/or operation described herein.

The database 10H can also contain contact information such as phone numbers, fax numbers, pager numbers, beeper numbers, e-mail addresses, hyperlinks to, and/or any other information which can facilitate contact between any of the parties described herein. The database 10H also includes electronic signature data and/or information for any of the parties, patients, providers, payers, and/or intermediaries, described herein for facilitating transactions, claim submissions, financial transactions, etc., by and/or between any of the above patients, providers, payers, and/or intermediaries.

The data and/or information in the database 10H can also include links to any other information, information sources, news sources, and/or other information and/or data which can or may be utilized by the present invention and/or by any of the patients, providers, payers, intermediaries and/or any other users of the present invention.

The database 10H can also contain data and/or information regarding healthcare news, healthcare developments, healthcare discoveries, etc., for and including the medical field, surgical field, psychological field, dental field, nutrition field, fitness field, etc., and/or any other healthcare field or fields.

The database 10H, in the preferred embodiment, can also contain video and/or audio files which can be utilized for training of healthcare professionals as well as for providing general information to any user of the present invention. In this manner, and as will be described hereinbelow, the apparatus 100 can be utilized as a simulator for providing training in medical diagnosing, medical training, surgical training, psychiatric training, psychological training, dental training, oral surgery training, therapist training, and/or for training any of the healthcare providers described herein and/or envisioned.

For example, the present invention can be utilized to provide a medical doctor with a set of symptoms, evaluate the diagnosis and treatment prescribed and provide follow-up patient conditions which may or may not call for the medical doctor to re-evaluate his or her diagnosis and/or treatment. In a similar fashion, the present invention can be used for training and continuing education and training for any of the healthcare providers described herein and/or otherwise envisioned utilizing the present invention.

The database 10H can also contain data and/or information restricting access by any of the providers, payers, patients, intermediaries, and/or other users, to any of the data and/or information stored in the database 10H.

The database 10H can also contain information correlating symptoms and/or conditions with diagnoses, prognoses, and/or treatments, treatment methods, procedures, etc. The database 10H also contains any and/or all information needed and/or desired for facilitating the processing of symptoms, conditions, medical histories, family histories, and other information, in order to arrive at diagnoses and/or prognoses, treatments, prescriptions, procedures and/or any other healthcare and/or healthcare-related information.

The database 10H can also contain statistical and/or other probabilistic and/or mathematical information for assigning and/or correlating certain levels and/or estimates for any and/or all of the information, diagnoses, prognoses, treatments, procedures, and/or any other information processed and/or generated by the central processing computer 10 and/or the apparatus 100.

The database 10H, in the preferred embodiment, can be a database which may include individual databases or collections of databases, with each database being designated to store any and all of the data and/or information described herein. The database 10H, or collection of databases, may be updated by each of the respective patients, providers, payers, users, and/or intermediaries, and/or by any other third party, in real-time, and/or via dynamically linked database management techniques.

The data and/or information stored in the database 10H can also be updated by and/or dynamically linked to, various external sources, including but not limited to news services, research publications, research facilities, healthcare laboratories, providers of healthcare goods and/or services, pharmaceutical companies, research institutions, schools, or any manufacturers, providers, sellers, service entities or servicing individuals, of or for any of the pacemakers 60, pacemaker monitors 65, defibrillators, and/or defibrillator monitors 75, described herein as being utilized in connection with the apparatus 100 of the present invention. The database 10H will contain any and all information deemed necessary and/or desirable for providing all of the processing and/or services and/or functions described herein.

The data and/or information which is contained and/or stored in the database 10, as well as any of the other databases 20H, 30H, 40, and 50H, described herein can be obtained from the various patients, individuals, providers, payers, and/or intermediaries, of from any manufacturers, providers, sellers, service entities or servicing individuals, of or for any of the pacemakers 60, pacemaker monitors 65, defibrillators, and/or defibrillator monitors 75, described herein, who or which utilize and/or who or which are serviced by the present invention. For example, the respective patients, providers, payers, intermediaries, and/or other users, could fill out questionnaires, forms, narratives, claim forms, and/or any other information medium, in written form, electronically, and/or otherwise.

Data and/or information stored in the database 10H as well as any of the other databases described herein can be updated by multiple parties. For example, a patient may provide medical history for his or her individual file; his or her medical doctor can update the medical history information for the patient upon examining and/or treating him or her. The payer may also update the file with any associated payment or payment-related information. Should the patient go to another doctor or different type of doctor, all previous information would be available for, and can be updateable by, the next doctor.

The database 10H can also include statistical data and/or information regarding diagnoses, and/or alternate diagnoses, treatment success, treatment failure, as well as statistical data and/or information regarding misdiagnoses. The database 10H also contains data and/or information regarding experimental treatments as well as statistical information regarding same, successes of same and failures of same.

In any and/or all of the embodiments described herein, any of the data and/or information which is or which may be stored in the database 10H, and/or any of the other databases described herein, can be utilized and/or can appear in any of the reports, diagnostic reports, treatment reports, evaluation reports, provider reports, payer reports, patient reports, training reports, and/or any other reports, described herein.

In a preferred embodiment, each of the apparatus 100, or any one or more of the central processing computer(s) 10, the provider computer(s) 20, the payer computers(s) 30, the patient computer(s) 40, and the intermediary computer(s) 50, or any one or more of the pacemakers 60, the pacemaker monitors 65, the defibrillators 70, or the defibrillator monitors 75, can be provided with appropriate hardware and/or software for performing any of the functions which are typically provided by programmer device used in the industry by service representatives, providers, nurses, or any other individuals who are trained to program, update, upgrade, calibrate, service, or otherwise perform functions on or regarding a pacemaker, a pacemaker monitor, a defibrillator, or a defibrillator monitor, including, but not limited to, communicating with a respective pacemaker, pacemaker monitor, defibrillator, or defibrillator monitor, via a wireless communication link, via RF, or via telemetry, or via the Internet and/or the World Wide Web, programming, changing, or modifying, programmable features of a respective pacemaker, pacemaker monitor, defibrillator, or defibrillator monitor, monitoring diagnostics, including, but not limited to, stored diagnostics, real-time diagnostics, detecting failures, malfunctions, or states of disrepair of, a respective pacemaker, pacemaker monitor, defibrillator, or defibrillator monitor, performing diagnostic routines to diagnose failures, malfunctions, or states of disrepair of, a respective pacemaker, pacemaker monitor, defibrillator, or a defibrillator monitor, trouble shooting problems with, in, or associated with, a respective pacemaker, pacemaker monitor, defibrillator, or defibrillator monitor, displaying real-time EKG/EGM data and/or information with or without markers for a respective, performing firmware or software upgrades to a respective pacemaker, pacemaker monitor, defibrillator, or defibrillator monitor, performing firmware or software upgrades to a respective pacemaker, pacemaker monitor, defibrillator, or defibrillator monitor, wirelessly, and to communicate or transmit any data and/or information regarding, or any functions performed on or regarding, or any follow-up information, regarding a respective pacemaker, pacemaker monitor, defibrillator, or defibrillator monitor, to the central processing computer 10 and to store same in the database 10H, or to transmit same to any of the herein-described the provider computer(s) 20, the payer computers(s) 30, the patient computer(s) 40, and the intermediary computer(s) 50, and to store same in their respective database(s). The database 10H can contain or information, including any software routines or algorithms for performing any of the herein-described functions or functionality.

In a preferred embodiment, each of the apparatus 100, or any one or more of the central processing computer(s) 10, the provider computer(s) 20, the payer computers(s) 30, the patient computer(s) 40, and the intermediary computer(s) 50, can also be programmed to generate an insurance claim or any other claim for payment of any bill for service and to transmit same to a respective payer computers 30, provider computer 20, patient computer 40, or intermediary computer 50. The database 10H can contain or information, including any software routines or algorithms for performing any of the herein-described functions or functionality.

In a preferred embodiment, any one or more of the patient computer(s) 40, or any one or more of the pacemakers 60, the pacemaker monitors 65, the defibrillators 70, or the defibrillator monitors 75, can be equipped with a global positioning device for ascertaining the position or location of same, and any or one or more of the central processing computer(s) 10, the provider computer(s) 20, the payer computers(s) 30, the patient computer(s) 40, and the intermediary computer(s) 50, or any one or more of the pacemakers 60, the pacemaker monitors 65, the defibrillators 70, or the defibrillator monitors 75, can have its software upgraded or uploaded wirelessly or via a wired hook-up or connection. The database 10H can contain or information, including any software routines or algorithms for performing any of the herein-described functions or functionality.

In a preferred embodiment, each of the apparatus 100, or any one or more of the central processing computer(s) 10, the provider computer(s) 20, the payer computers(s) 30, the patient computer(s) 40, and the intermediary computer(s) 50, or any one or more of the pacemakers 60, the pacemaker monitors 65, the defibrillators 70, or the defibrillator monitors 75, can be provided with appropriate hardware and/or software for receiving, storing, downloading, and/or transmitting to a monitoring company, entity, computer, communication device, or any other suitable or appropriate device, any pacemaker or defibrillator, or respective pacemaker monitor or defibrillator monitor, functional information, diagnostic information, alerts, malfunction alerts, diagnostics alerts, failure, malfunction, or state of disrepair, alerts, service or maintenance alerts, or any other alerts, at any frequency or time interval, once a day, or any desired number of time a day, or at any other desired times or time intervals. Any data and/or information can then be transmitted to an intermediary or third party such a pacemaker or defibrillator monitoring company or entity. The database 10H can contain or information, including any software routines or algorithms for performing any of the herein-described functions or functionality.

In a preferred embodiment, each of the apparatus 100, or any one or more of the central processing computer(s) 10, the provider computer(s) 20, the payer computers(s) 30, the patient computer(s) 40, and the intermediary computer(s) 50, or any one or more of the pacemakers 60, the pacemaker monitors 65, the defibrillators 70, or the defibrillator monitors 75, can be provided with appropriate hardware and/or software for linking a respective pacemaker 60, pacemaker monitor 65, defibrillator 70, and/or defibrillator monitor 75, to any of the herein-described central processing computer(s) 10, the provider computer(s) 20, the payer computers(s) 30, the patient computer(s) 40, and the intermediary computer(s) 50, regarding of regardless of where the respective pacemaker 60, pacemaker monitor 65, defibrillator 70, and/or defibrillator monitor 75 and/or wirelessly, for facilitating the ability to ask symptom questions of the patient using voice synthesizing techniques or other appropriate or suitable techniques, and/or for monitoring real-time diagnostic information and/or stored diagnostic information, for detecting and/or treating Brady arrhythmias by both sensing and pacing any or all chambers of the heart, for performing an Anti-Tachycardia Pace of or for any or all chambers of the heart, for detecting and/or modulating a pacing rate based on feedback from the patient, from a respective pacemaker or defibrillator, or from any appropriate device, monitor, or interface, or computer or communication device which may be used for performing said functionality, for measuring and/or sensing the Amplitude Threshold of each chamber, for measuring the Pacing Threshold of each chamber, for measuring the Lead Impedance of each chamber and of any other or additional, or extra, devices, monitors, or interfaces, which can or may be attached to the respective pacemaker or defibrillator, for measuring shocking coil impedance, for overcompensation of the pacing rate(s) for, or attributed to, sudden rate drops, and/or for measuring diagnostics such as Heart Rate Variability, Time spent in arrhythmias, and/or Frequency of each arrhythmia. The database 10H can contain or information, including any software routines or algorithms for performing any of the herein-described functions or functionality.

In a preferred embodiment, each of the apparatus 100, or any one or more of the central processing computer(s) 10, the provider computer(s) 20, the payer computers(s) 30, the patient computer(s) 40, and the intermediary computer(s) 50, or any one or more of the pacemakers 60, the pacemaker monitors 65, the defibrillators 70, or the defibrillator monitors 75, can be provided with appropriate hardware and/or software for performing the programming of programmable Pacing features for pacemakers, including, but not limited to, programming, adjusting, or calibrating, Pacing Mode, Rate Modulation, Refractory Periods, and/or Power Output of each Pace including voltage, current, and pulse width, and for performing the programming of programmable defibrillating features for defibrillators, including, but not limited to, programming, adjusting, or calibrating, Shocking Power Output, Anti-Tachycardia Pacing Schemes, Detection Algorithms, and Anti-Tachycardia Discrimination Algorithms, as well as for performing Detection Algorithms, and Anti-Tachycardia Discrimination Algorithms or any other algorithms which can be performed by or on a respective pacemaker or defibrillator. The database 10H can contain or information, including any software routines or algorithms for performing any of the herein-described functions or functionality.

The database 10H can contain information for accessing or connecting to, or a link or a hyperlink to or for, any of the herein-described pacemakers 60, pacemaker monitors 65, defibrillators 70, and defibrillator monitors 75, for facilitating a remote accessing of a respective pacemaker 60, pacemaker monitor 65, defibrillator 70, or defibrillator monitor 75, for allowing a provider, a payer, an intermediary, a third party, a caregiver, a relative, or any other authorized individual or entity, or the patient or individual himself or herself, to control an operation of, to activate, to de-activate, to enable, or to disable, a respective pacemaker 60, pacemaker monitor 65, defibrillator 70, or defibrillator monitor 75, to monitor an operation of, to transmit a signal, data, or information, to or to obtain or receive a signal, data, or information, from a respective pacemaker 60, pacemaker monitor 65, defibrillator 70, or defibrillator monitor 75, and/or to program, re-program, calibrate, set or reset, and/or to perform a diagnostic function on or regarding, a respective pacemaker 60, pacemaker monitor 65, defibrillator 70, or defibrillator monitor 75, thereby providing a healthcare record, file, or history, or a medical record, file, or history, via which, or by means of which, the respective provider, payer, intermediary, third party, caregiver, relative, any other authorized individual or entity, or the patient or individual himself or herself, can perform any of the above-described, or any other of the herein-described, operations or functions regarding or relating to a respective pacemaker 60, pacemaker monitor 65, defibrillator 70, or defibrillator monitor 75.

In this manner, the apparatus 100 and method of the present invention can provide for the linking of a respective pacemaker 60, pacemaker monitor 65, defibrillator 70, or defibrillator monitor 75, to an electronic healthcare record, file, or history, or to an electronic medical record, file, or history.

The central processing computer 10 also includes an output device 10I for output any data, information, report, etc., described herein. In the preferred embodiment, the output device 10I can be a printer, a display, a transmitter, a modem, and/or any other device which can be used to output data.

Any of the data and/or information for any of the patients, individuals, providers, payers, and/or intermediaries, or for any of the manufacturers, providers, sellers, service entities or servicing individuals, of or for any of the pacemakers 60, pacemaker monitors 65, defibrillators, and/or defibrillator monitors 75, described herein, or for any of the pacemakers 60, pacemaker monitors 65, defibrillators, and/or defibrillator monitors 75, described herein, can be updated by different parities and which such updated data and/or information being made available to other respective parties so as to provide and ensure comprehensive and up-to-date healthcare and healthcare-related information.

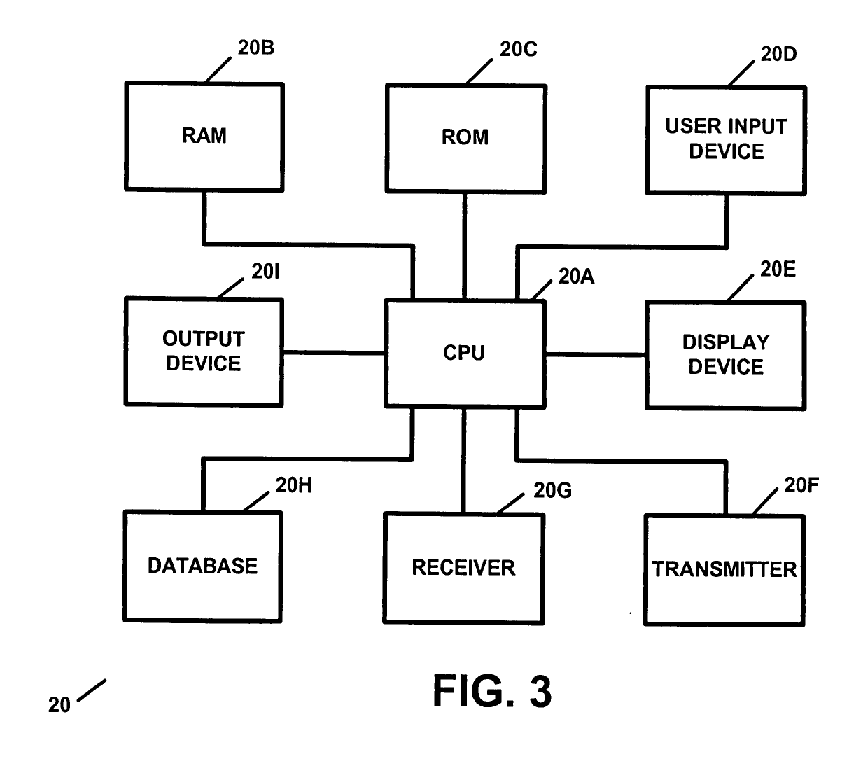

FIG. 3 illustrates the provider communication device 20, in block diagram form. The provider communication device 20, in the preferred embodiment, can be personal computer, a network computer or computer system, or any other computer or communication device, which is utilized as a provider computer 20. In the preferred embodiment, the provider communication device 20 includes a central processing unit or CPU 20A, which in the preferred embodiment, is a microprocessor. The CPU 20A may also be a microcomputer, a minicomputer, a macro-computer, and/or a mainframe computer, depending upon the application.

The provider communication device 20 also includes a random access memory device(s) 20B (RAM) and a read only memory device(s) 20C (ROM), each of which is connected to the CPU 20A, a user input device 20D, for entering data and/or commands into the provider communication device 20, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electrocardiograms, blood-sugars monitors, etc., or any of the herein-described pacemakers 60, pacemaker monitors 65, defibrillators 70, or defibrillator monitors 75, if desired, can also connected to or otherwise linked to the CPU 20A, either directly or indirectly via any of the herein-described computers 10, 20, 30, 40, and 50.

The user input device 20D can also acquire, receive, generate and/or provide, data which can be entered by a user or individual and/or can be a device which can acquire, receive, generate and/or provide, digital data such as medical, healthcare, bio-metric, physiological, and/or any other kind of healthcare data and/or healthcare-related data. For example, a user input device 20D can be a keyboard for allowing a user to input information into the provider communication device and/or a user input device can, for example, by a heart rate monitor or EKG machine, etc., or any of the herein-described pacemakers 60, pacemaker monitors 65, defibrillators 70, or defibrillator monitors 75, if desired, which can receive information from a patient or individual and generate a digital signal, digital data, analog data, and/or any other signal, data, and/or information, which is representative of the patient's or individual's heart rate, pulse rate, and/or cardiac activity.

The provider communication device 20 also includes a display device 20E for displaying data and/or information to a user or operator.

The provider communication device 20 also includes a transmitter(s) 20F, for transmitting signals and/or data and/or information to any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention. The provider communication device 20 also includes a receiver 20G, for receiving signals and/or data and/or information from any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention.

The provider communication device 20 also includes a database(s) 20H. The database 20H can contain and/or be linked to any of the data and/or information described herein as being stored in the database 10H.

The provider communication device 20 also includes an output device 201 for output any data, information, report, etc., described herein. In the preferred embodiment, the output device 201 can be a printer, a display, a transmitter, a modem, and/or any other device which can be used to output data.

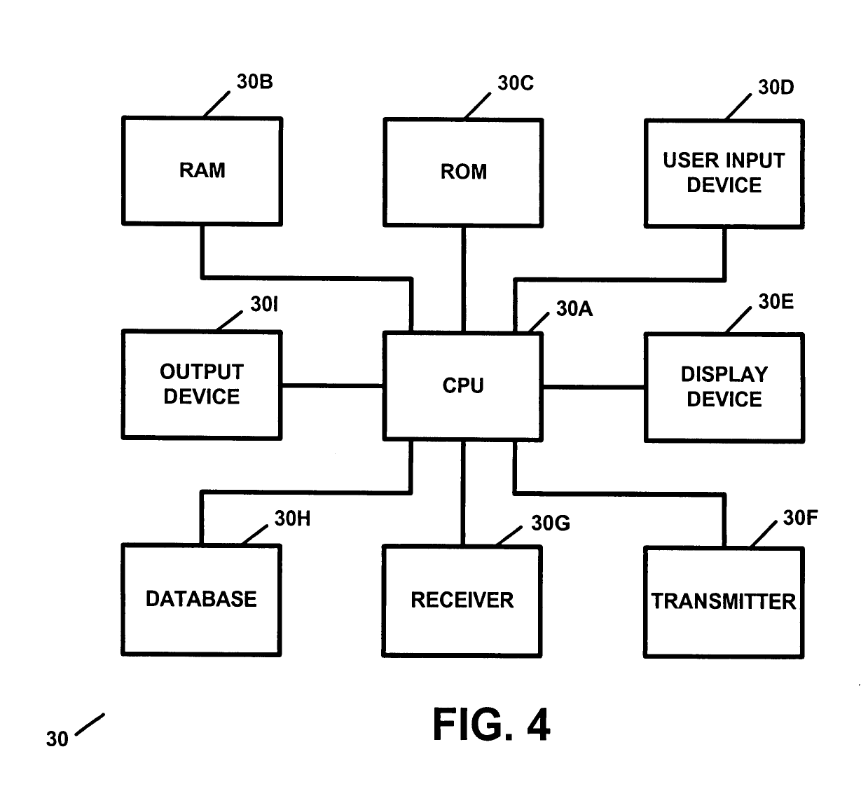

FIG. 4 illustrates the payer communication device 30, in block diagram form. The payer communication device 30, in the preferred embodiment, can be personal computer, a network computer or computer system, or any other computer or communication device, which is utilized as a payer computer 30. In the preferred embodiment, the payer communication device 30 includes a central processing unit or CPU 30A, which in the preferred embodiment, is a microprocessor. The CPU 30A may also be a microcomputer, a minicomputer, a macro-computer, and/or a mainframe computer, depending upon the application.

The payer communication device 30 also includes a random access memory device(s) 30B (RAM) and a read only memory device(s) 30C (ROM), each of which is connected to the CPU 30A, a user input device 30D, for entering data and/or commands into the payer communication device 30, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, which input device(s) are also connected to the CPU 30A. The payer communication device 30 also includes a display device 30E for displaying data and/or information to a user or operator.

The payer communication device 30 also includes a transmitter(s) 30F, for transmitting signals and/or data and/or information to any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention. The payer communication device 30 also includes a receiver 30G, for receiving signals and/or data and/or information from any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention.

The payer communication device 30 also includes a database(s) 30H. The database 30H can contain and/or be linked to any of the data and/or information described herein as being stored in the database 10H.

The payer communication device 30 also includes an output device 301 for output any data, information, report, etc., described herein. In the preferred embodiment, the output device 301 can be a printer, a display, a transmitter, a modem, and/or any other device which can be used to output data.

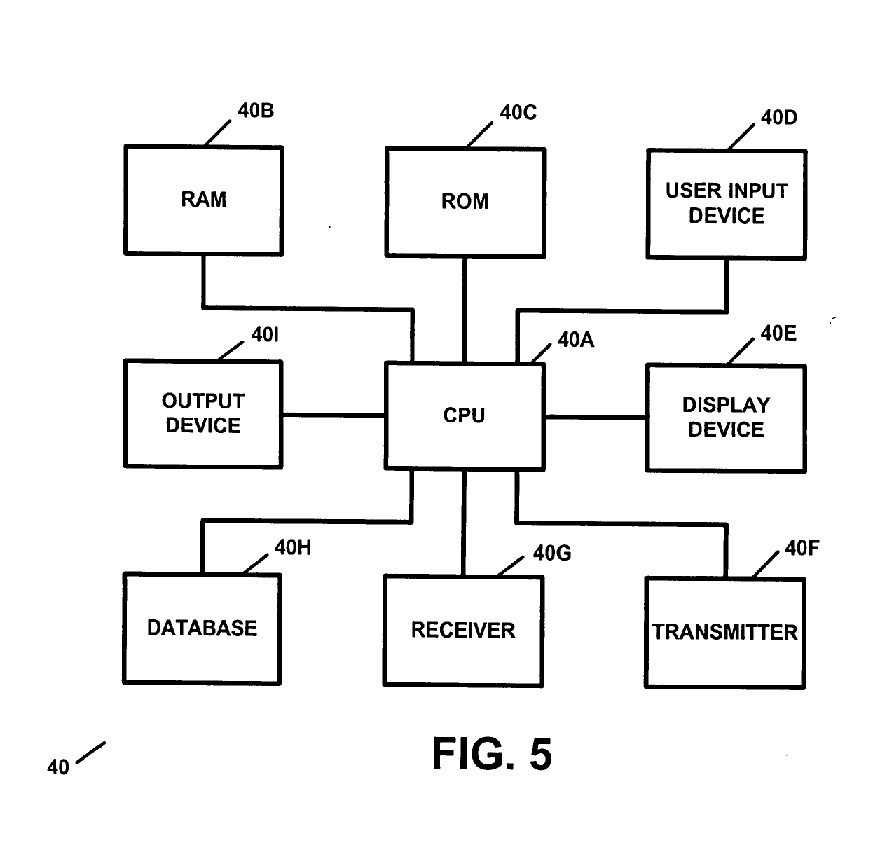

FIG. 5 illustrates the patient communication device 40, in block diagram form. The patient communication device 40, in the preferred embodiment, can be personal computer, a network computer or computer system, or any other computer or communication device, which is utilized as a patient computer. In the preferred embodiment, the patient communication device 40 includes a central processing unit or CPU 40A, which in the preferred embodiment, is a microprocessor. The CPU 40A may also be a microcomputer, a minicomputer, a macro-computer, and/or a mainframe computer, depending upon the application.

The patient communication device 40 also includes a random access memory device(s) 40B (RAM) and a read only memory device(s) 40C (ROM), each of which is connected to the CPU 40A, a user input device 40D, for entering data and/or commands into the patient communication device 40, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electro-cardiograms, blood-sugars monitors, etc., or for inputting and/or entering data and/or information from any of the herein-described pacemakers, pacemaker monitors 65, defibrillators 70, and/or defibrillator monitors 75, if desired, which input device(s) are also connected to the CPU 40A.

The user input device 40D can also acquire, receive, generate and/or provide, data which can be entered by a user or individual and/or can be a device which can acquire, receive, generate and/or provide, digital data such as medical, healthcare, bio-metric, physiological, and/or any other kind of healthcare data and/or healthcare-related data. For example, a user input device 20D can be a keyboard for allowing a user to input information into the patient communication device and/or a user input device can, for example, by a heart rate monitor, an EKG machine, a pacemaker 60, a pacemaker monitor 65, a defibrillator 70, or a defibrillator monitor 75, which can receive or obtain information from or regarding a patient or individual and generate a digital signal, digital data, analog data, and/or any other signal, data, and/or information, which is representative of the patient's or individual's heart rate, pulse rate, and/or cardiac activity.

The patient communication device 40 also includes a display device 40E for displaying data and/or information to a user or operator.

The patient communication device 40 also includes a transmitter(s) 40F, for transmitting signals and/or data and/or information to any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention. The patient communication device 40 also includes a receiver 40G, for receiving signals and/or data and/or information from any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention.

The patient communication device 40 also includes a database(s) 40H. The database 40H can contain and/or be linked to any of the data and/or information described herein as being stored in the database 10H.

The patient communication device 40 also includes an output device 401 for output any data, information, report, etc., described herein. In the preferred embodiment, the output device 401 can be a printer, a display, a transmitter, a modem, and/or any other device which can be used to output data.

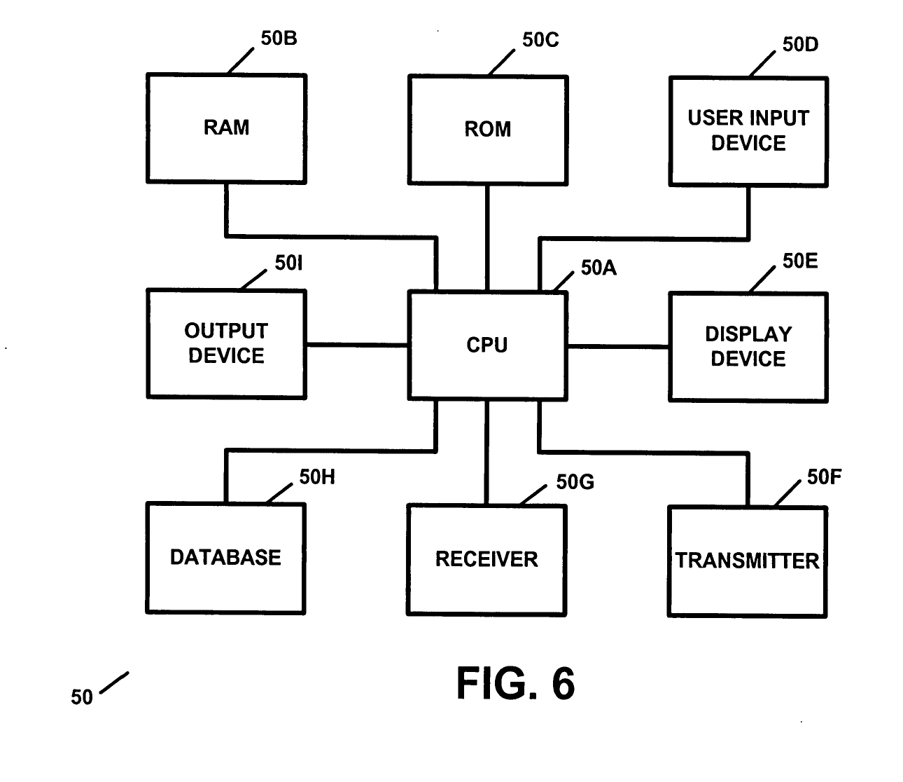

FIG. 6 illustrates the intermediary computer 50, in block diagram form. The intermediary computer 50, in the preferred embodiment, can be personal computer, a network computer or computer system, or any other computer or communication device, which is utilized as an intermediary computer 50. In the preferred embodiment, the intermediary computer 50 includes a central processing unit or CPU 50A, which in the preferred embodiment, is a microprocessor. The CPU 50A may also be a microcomputer, a minicomputer, a macro-computer, and/or a mainframe computer, depending upon the application.

The intermediary computer 50 also includes a random access memory device(s) 50B (RAM) and a read only memory device(s) 50C (ROM), each of which is connected to the CPU 50A, a user input device 50D, for entering data and/or commands into the intermediary computer 50, which includes any one or more of a keyboard, a scanner, a user pointing device, such as, for example, a mouse, a touch pad, and/or an audio input device and/or a video input device, and/or any device, electronic and/or otherwise which can be utilized for inputting and/or entering healthcare data and/or information, for example pulse rate monitors, blood pressure monitors, electrocardiograms, blood-sugars monitors, etc., or any of the herein-described pacemakers 60, pacemaker monitors 65, defibrillators 70, or defibrillator monitors 75, if desired, if desired, which input device(s) are or can also connected to the CPU 50A. The intermediary computer 50 also includes a display device 50E for displaying data and/or information to a user or operator.

The intermediary computer 50 also includes a transmitter(s) 50F, for transmitting signals and/or data and/or information to any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention. The intermediary computer 50 also includes a receiver 50G, for receiving signals and/or data and/or information from any one or more of the central processing computer(s) 10, the provider computer(s) 20, the payer computers(s) 30, the patient computer(s) 40, and the intermediary computer(s) 50, or to any one or more of the pacemakers 60, the pacemaker monitors 65, the defibrillators 70, or the defibrillator monitors 75, which may be utilized in conjunction with the present invention.

The provider communication device 50 also includes a database(s) 50H. The database 50H can contain and/or be linked to any of the data and/or information described herein as being stored in the database 10H.

The intermediary communication device 50 also includes an output device 501 for output any data, information, report, etc., described herein. In the preferred embodiment, the output device 501 can be a printer, a display, a transmitter, a modem, and/or any other device which can be used to output data.

In any and/or all of the embodiments described herein, any one of the central processing computers 10, the provider communication devices 20, the payer communication devices 30, the patient communication devices 40, and/or the intermediary communication devices 50, can include input devices (not shown) for facilitating the data entry of a patients vital signs and or other medical data such as, but not limited to, pulse rate, blood pressure, blood-sugar level, etc., and any other data and or information which can be input into the respective computer and/or communication device and be transmitted to the central processing computer 10 consistent with the utilization of the present invention as described herein.

The apparatus and method of the present invention can be utilized in numerous preferred embodiments in order to provide a vast array of healthcare and healthcare-related services for any one or more of the various parties described herein relating to, or regarding the monitoring, controlling, servicing, and/or maintenance, of the pacemakers 60, pacemaker monitors 65, defibrillators 70, and/or defibrillator monitors 75, described herein.

While certain of the preferred embodiments may be described with regards to utilization by a particular party, it is important to note that any patient, user, provider, payer, and/or intermediary may utilize the present invention in the same, similar and/or analogous manner. For example, a preferred embodiment for determining and/or ascertaining a medical diagnosis can be described as being utilized by a treating physician as well as be utilized by a provider to verify and/or check a diagnosis as well as by a patient or other user or individual in order to perform a self-diagnosis or double check a doctors diagnosis. In the same manner, any other preferred embodiment and/or other uses of the present invention can be utilized by any of the parties described herein.

In a preferred embodiment, the apparatus 100 and method of the present invention can be utilized by any authorized provider of the individual or patient, payer of the individual or patient, intermediary, third party, caregiver, relative, any other authorized individual or entity, or the patient or individual himself or herself, in order to access or link to, or be connected to, or be brought into communication with, a respective pacemaker 60, pacemaker monitor 65, defibrillator 70, or defibrillator monitor 75, of or associated with an individual or patient, via the individual's or patient's healthcare record, file, or history, or medical record, file, or history, in order to allow the respective and authorized provider of the individual or patient, payer of the individual or patient, intermediary, third party, caregiver, relative, any other authorized individual or entity, or the patient or individual himself or herself, to control an operation of, to activate, to de-activate, to enable, or to disable, the respective pacemaker 60, pacemaker monitor 65, defibrillator 70, or defibrillator monitor 75, to monitor an operation of, to transmit a signal, data, or information, to or to obtain or receive a signal, data, or information, from the respective pacemaker 60, pacemaker monitor 65, defibrillator 70, or defibrillator monitor 75, and/or to program, re-program, calibrate, set or reset, and/or to perform a diagnostic function on or regarding, the respective pacemaker 60, pacemaker monitor 65, defibrillator 70, or defibrillator monitor 75.

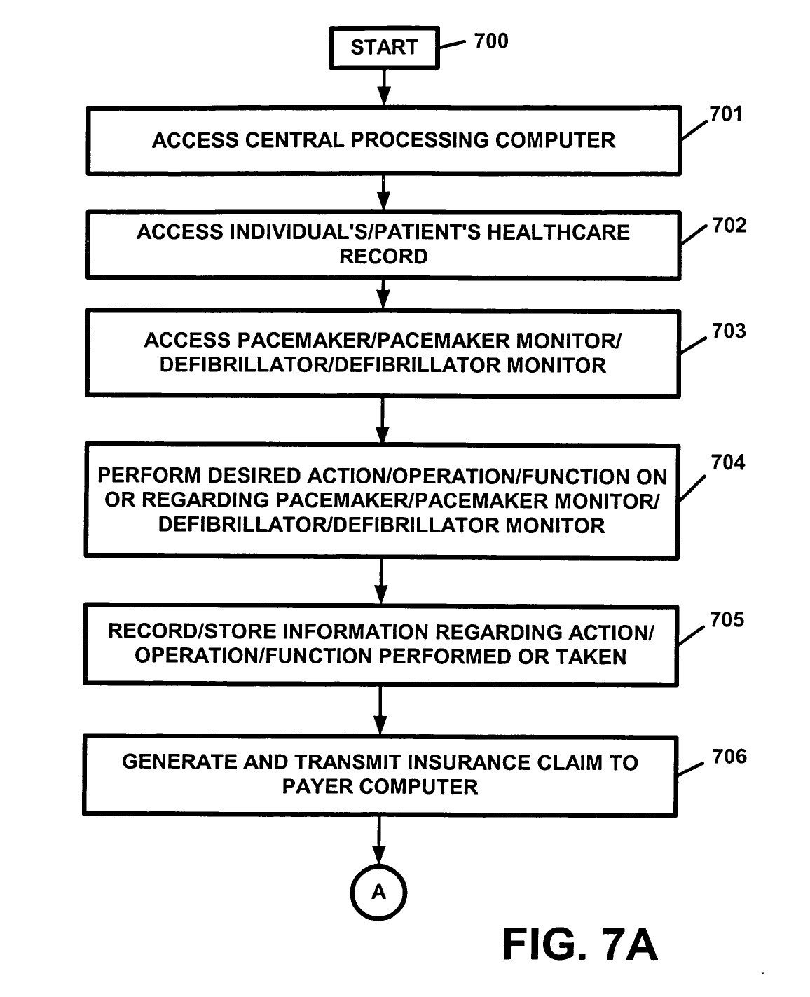

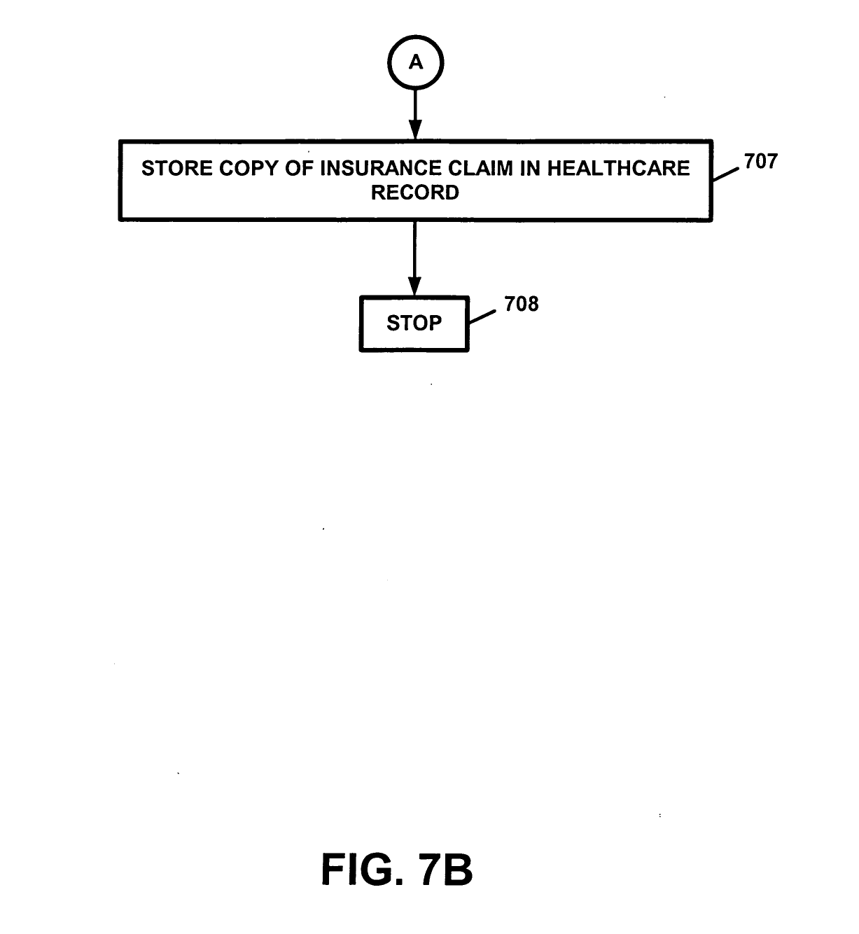

FIGS. 7A and 7B illustrate a preferred embodiment method of using the apparatus 100 and method of the present invention, in flow diagram form. With reference to FIGS. 7A and 7B, the operation of the apparatus 100 commences at Step 700. At step 701, the respective and authorized provider of the individual or patient, payer of the individual or patient, intermediary, third party, caregiver, relative, any other authorized individual or entity, or the patient or individual himself or herself, can access the central processing computer 10 using his, her, or its respective provider computer 20, payer computers 30, patient computer 40, or intermediary computer 50.

At step 702, the respective provider, payer, patient, intermediary, third party, caregiver, relative, any other authorized individual or entity, or the patient or individual himself or herself, can access the individual's or patient's healthcare record, file, or history, or medical record, file, or history, and link or hyperlink to, or use any other information provided in the individual's or patient's healthcare record, file, or history, or medical record, file, or history, in order to link to or hyperlink to, so as to, and in order to, access, the individual's or patient's pacemaker 60, pacemaker monitor 65, defibrillator 70, or defibrillator monitor 75. In the case of accessing a respective pacemaker 60, the respective provider, payer, patient, intermediary, third party, caregiver, relative, any other authorized individual or entity, or the patient or individual himself or herself, can access the respective pacemaker 60 directly or indirectly via the respective pacemaker monitor 65. In the case of accessing a respective defibrillator 70, the respective provider, payer, patient, intermediary, third party, caregiver, relative, any other authorized individual or entity, or the patient or individual himself or herself, can access the respective defibrillator 70 directly or indirectly via the respective defibrillator monitor 75.

At step 703, the respective provider, payer, patient, intermediary, third party, caregiver, relative, any other authorized individual or entity, or the patient or individual himself or herself, can access the individual's or patient's pacemaker 60, pacemaker monitor 65, defibrillator 70, or defibrillator monitor 75.

At step 704, the respective provider, payer, patient, intermediary, third party, caregiver, relative, any other authorized individual or entity, or the patient or individual himself or herself, can then, via his, her, or its respective provider computer 20, payer computers 30, patient computer 40, or intermediary computer 50, perform any desired function or operation in order to control an operation of, to activate, to de-activate, to enable, or to disable, the respective pacemaker 60, pacemaker monitor 65, defibrillator 70, or defibrillator monitor 75, to monitor an operation of, to transmit a signal, data, or information, to or to obtain or receive a signal, data, or information, from the respective pacemaker 60, pacemaker monitor 65, defibrillator 70, or defibrillator monitor 75, and/or to program, re-program, calibrate, set or reset, and/or to perform a diagnostic function or a repair function on or regarding, the respective pacemaker 60, pacemaker monitor 65, defibrillator 70, or defibrillator monitor 75.

At step 704, the respective provider, payer, patient, intermediary, third party, caregiver, relative, any other authorized individual or entity, or the patient or individual himself or herself, can also, or in addition, perform any other function or operation which can typically or usually be performed on, or regarding or related to, or associated with a pacemaker 60, pacemaker monitor 65, defibrillator 70, or defibrillator monitor 75.

At step 704, the respective provider, payer, patient, intermediary, third party, caregiver, relative, any other authorized individual or entity, or the patient or individual himself or herself, can also ascertain the position or location of the respective pacemaker 60, pacemaker monitor 65, defibrillator 70, or defibrillator monitor 75, can upgrade or upload software to the respective pacemaker 60, pacemaker monitor 65, defibrillator 70, can receive, store, download, and/or transmit information to a monitoring company, entity, computer, communication device, or any other suitable or appropriate device, for or regarding any pacemaker or defibrillator, or respective pacemaker monitor or defibrillator monitor, can obtain functional or operational information, diagnostic information, information regarding a malfunction, a failure, a state of disrepair, a diagnostic code, an alert, a service or maintenance alert, or any other alert(s), can monitor real-time or stored diagnostic data or information, detect and/or treat Brady arrhythmias by both sensing and pacing any or all chambers of the heart, can perform an Anti-Tachycardia Pacing of or for any or all chambers of the heart, can detect and/or modulate a pacing rate based on feedback from the patient or from the respective pacemaker 60, pacemaker monitor 65, defibrillator 70, or defibrillator monitor 75, can measure and/or sense the Amplitude Threshold of each chamber, can measure the Pacing Threshold of each chamber, can measure the Lead Impedance of each chamber and of any other or additional, or extra, devices, monitors, or interfaces, which can or may be attached to or linked to or with the respective pacemaker 60, pacemaker monitor 65, defibrillator 70, or defibrillator monitor 75, can measure shocking coil impedance, can provide compensate or overcompensation of a pacing rate(s) for, or attributed to, sudden rate drops, can measure diagnostics such as Heart Rate Variability, Time spent in arrhythmias, and/or Frequency of each arrhythmia, can program programmable Pacing features for pacemakers, including, but not limited to, programming, adjusting, or calibrating, Pacing Mode, Rate Modulation, Refractory Periods, and/or Power Output of each Pace including voltage, current, and pulse width, can program programmable defibrillating features for defibrillators, including, but not limited to, programming, adjusting, or calibrating, Shocking Power Output, Anti-Tachycardia Pacing Schemes, Detection Algorithms, and Anti-Tachycardia Discrimination Algorithms, can perform Detection Algorithms, Anti-Tachycardia Discrimination Algorithms, or any other algorithms which can be performed by or on the respective pacemaker 60, pacemaker monitor 65, defibrillator 70, or defibrillator monitor 75, or can perform any other operation, action, or function, on or regarding or relating to, the respective pacemaker 60, pacemaker monitor 65, defibrillator 70, or defibrillator monitor 75.

At step 704, the respective provider, payer, patient, intermediary, third party, caregiver, relative, any other authorized individual or entity, or the patient or individual himself or herself, can perform any number or combination of actions, functions, or operations, on, for, regarding, or relating to, the respective pacemaker 60, pacemaker monitor 65, defibrillator 70, or defibrillator monitor 75.

At step 705, the central processing computer 10 can store any data and/or information regarding any and/or all of the action(s), function(s), or operation(s), performed by the respective provider, payer, patient, intermediary, third party, caregiver, relative, any other authorized individual or entity, or the patient or individual himself or herself, on, for, regarding, or relating to, the respective pacemaker 60, pacemaker monitor 65, defibrillator 70, or defibrillator monitor 75, in the individual's or the patient's healthcare record, file, or history, or medical record, file, or history, in the database 10H. At step 705, any data and/or information regarding any and/or all of the action(s), function(s), or operation(s), performed by the respective provider, payer, patient, intermediary, third party, caregiver, relative, any other authorized individual or entity, or the patient or individual himself or herself, on, for, regarding, or relating to, the respective pacemaker 60, pacemaker monitor 65, defibrillator 70, or defibrillator monitor 75, can also be stored in the respective database 20H, 30H, 40H, or 50H, of the respective provider computer 20, payer computers 30, patient computer 40, or intermediary computer 50.

At step 706, in instances where the respective provider, payer, patient, intermediary, third party, caregiver, or any other authorized individual or entity, is to be paid for his, her, or its, services performed for or on behalf of the individual or patient or relating to his, her, or its services rendered for or on behalf of the individual or patient, at any time as well as during steps 701-705, or at any other time, for or otherwise on behalf of the individual or patient, the central processing computer 10, can generate an insurance claim or insurance claim form which is suitable for automatic submission to the payer or healthcare insurer of or associated with the individual or patient. At step 706, the central processing computer will generate the insurance claim or insurance claim form and will transmit same to the payer computer 30 of or associated with the individual's or the patient's payer, healthcare insurance company, or healthcare insurer.

At step 707, the central processing computer 10 can also store a copy of the insurance claim or insurance claim form sent to the individual's or the patient's payer, healthcare insurance company, or healthcare insurer, along with any other desired information relating to same, in the individual's or the patient's healthcare record, file, or history, or medical record, file, or history, in the database 10H. At step 707, a copy of the insurance claim or insurance claim form sent to the individual's or the patient's payer, healthcare insurance company, or healthcare insurer, along with any other desired information relating to same, can also be stored in the respective database 20H, 30H, 40H, or 50H, of the respective provider computer 20, payer computer 30, patient computer 40, or intermediary computer 50.

Thereafter, the operation of the apparatus 100 will cease at step 708.

In another preferred embodiment, each of the pacemakers 60, the pacemaker monitors 65, the defibrillators 70, or the defibrillator monitors 75, can be programmed to generate and/or to send notification messages or alerts regarding the occurrence(s) of certain events or happenings. In this embodiment, each of the pacemakers 60, the pacemaker monitors 65, the defibrillators 70, or the defibrillator monitors 75, can be quipped with the needed, required, or desired, hardware and/or software for detecting the occurrence of the events or happenings, for generating the notification messages or alerts, and for transmitting the notification messages or alerts to a respective computer or communication device 20, 30, 40, or 50, of a respective the respective provider, payer, patient, intermediary, third party, caregiver, relative, any other authorized individual or entity, or the patient or individual himself or herself.

In the preferred embodiment, a respective pacemaker 60, pacemaker monitor 65, defibrillator 70, or defibrillator monitor 75, can detect and report on any occurrence, event, or happening, which warrants providing a notification or an alert to any provider, payer, patient, intermediary, third party, caregiver, relative, any other authorized individual or entity, or the patient or individual himself or herself, or any other user of the apparatus 100 of the present invention.

In the preferred embodiment, the occurrence, event, or happening, can, for example, be, but is not limited to, a detected malfunction, failure, or state of disrepair, of a respective pacemaker 60, pacemaker monitor 65, defibrillator 70, or defibrillator monitor 75, an individual or patient having received an electric shock, an individual or patient having received Tachy Arrhythmia Therapy such as Anti-Tachycardia Pacing, an increase in a pacing threshold of an individual or patient, an individual's or patient's pacing threshold having increased so much that the respective pacemaker 60, pacemaker monitor 65, defibrillator 70, or defibrillator monitor 75, is no longer capturing an individual's or patient's pacing impedance having increased or decreased by more than a recommended amount by a lead, an individual's or patient's sensing threshold having increased or decreased by more than a recommended amount or limit, an individual's or patient's sensing threshold having changed so much that the respective pacemaker 60, pacemaker monitor 65, defibrillator 70, or defibrillator monitor 75, is no longer sensing or operating correctly or with the required or desired degree or level of confidence, the individual or patient having experienced an arrhythmia which the respective pacemaker 60, pacemaker monitor 65, defibrillator 70, or defibrillator monitor 75, has detected and has declared to be an episode, that the individual's or patient's respective pacemaker 60, pacemaker monitor 65, defibrillator 70, or defibrillator monitor 75, has enacted a feature unique to that particular device, that the individual or patient has another device(s) that the respective pacemaker monitor 65 or defibrillator monitor 75 is monitoring and which has reached a certain threshold so as to declare an alert or an alert regarding the health and well-being of the individual or patient, that the individual or patient has had or has experienced a change in status for any of the symptoms that the respective pacemaker 60, pacemaker monitor 65, defibrillator 70, or defibrillator monitor 75 is monitoring or being used for, that the individual or patient, or any other authorized person, who has access to one of the respective pacemaker monitors 65 or defibrillator monitors 75, has declared to be an emergency thereby triggering an alerting of one or more, or all, of the parties involved in an individual's or patient's care, that the respective pacemaker 60, pacemaker monitor 65, defibrillator 70, or defibrillator monitor 75, has declared a malfunction or a state of disrepair, that a respective pacemaker monitor 65 or a defibrillator monitor 75 can no longer make contact with or communicate with a respective pacemaker 60 or defibrillator 70 thereby triggering a need to declare an emergency, and/or any other occurrence, event, or happening, which may warrant the providing of notice or an alert to any of the individuals or entities described herein as using the apparatus 100 of the present invention.

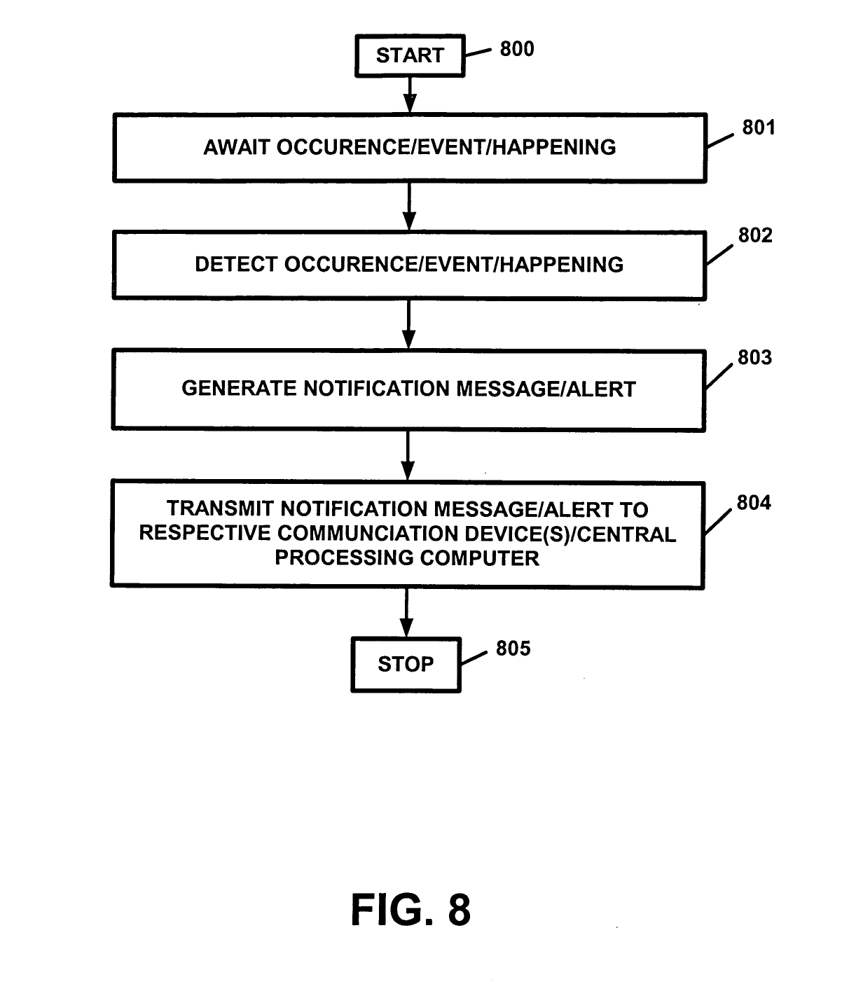

FIG. 8 illustrates another preferred embodiment method of using the present invention, in flow diagram form. In a preferred embodiment, any requests for notification messages or alerts can be pre-specified and/or can be pre-programmed or designed into the respective pacemaker 60, pacemaker monitor 65, defibrillator 70, or defibrillator monitor 75, at any time.

With reference to FIG. 8, the operation of the apparatus 100 will commence at step 800. At step 801, the apparatus 100 and/or the respective pacemaker 60, pacemaker monitor 65, defibrillator 70, or defibrillator monitor 75, of or associated with an individual or patient will await an occurrence or a happening of an occurrence, event, or happening, which has been pre-specified or pre-programmed as being a subject of a notification message or an alert. Upon the occurrence or happening of the occurrence, event, or happening, the respective pacemaker 60, pacemaker monitor 65, defibrillator 70, or defibrillator monitor 75, will detect same at step 802.

At step 803, the respective pacemaker 60, pacemaker monitor 65, defibrillator 70, or defibrillator monitor 75, will generate a notification message or an alert containing information identifying the occurrence, event, or happening, date and time of same, and any other information regarding same.

At step 804, the respective pacemaker 60, pacemaker monitor 65, defibrillator 70, or defibrillator monitor 75, will transmit the notification message or alert to one or more of the respective communication devices 20, 30, 40, or 50, associated with or used by any one or more of the respective individual or patient, a relative or caregiver of or for the individual or patient, as well as any provider(s), payer(s), or intermediary or intermediaries, associated with the individual or patient, the respective pacemaker 60, pacemaker monitor 65, defibrillator 70, or defibrillator monitor 75, monitoring company or individual, and/or any emergency medical services agency, thereby providing each with notice of the occurrence, event, or happening. At step 804, the respective pacemaker 60, pacemaker monitor 65, defibrillator 70, or defibrillator monitor 75, will transmit the notification message or alert to the central processing computer 10 and the central processing computer can store any information regarding same, and of the occurrence, event, or happening, in the database 10H and, in particular, in the healthcare record, file, or history, or the medical record, file, or history, of the individual or patient.

Thereafter, the operation of the apparatus 100 will cease at step 805. Thereafter, any provider, payer, intermediary, or the individual or patient, may also utilize the apparatus of the embodiment of FIGS. 7A and 7B in order to perform any desired actions, function, or operation.

In any and/or all of the embodiments described herein, the respective pacemaker 60, pacemaker monitor 65, defibrillator 70, or defibrillator monitor 75, can communicate in bi-directional manner with any one or more of the respective databases 10H, 20H, 30H, 40H, or 50H, of any of the respective central processing computer(s) 10, provider computer(s) 20, payer computer(s) 30, patient computer(s) 40, or intermediary computer(s) 50, and/or with any of the healthcare records, files, or histories, or medical records, files, or histories store therein or associated therewith.

In any and/or all of the embodiments described herein, the respective pacemaker 60, pacemaker monitor 65, defibrillator 70, or defibrillator monitor 75, can provide and transmit real-time electrogram or real-time EKG data and/or information, and/or any data and/or any data and/or information regarding any arrhythmia(s), feature activation(s), or respective pacemaker 60, pacemaker monitor 65, defibrillator 70, or defibrillator monitor 75, malfunction(s), failure(s), or state(s) of disrepair, to any one or more of the respective databases 10H, 20H, 30H, 40H, or 50H, of any of the respective central processing computer(s) 10, provider computer(s) 20, payer computer(s) 30, patient computer(s) 40, or intermediary computer(s) 50, for storage thereat or therein, and/or for storage in any of the healthcare records, files, or histories, or medical records, files, or histories, stored therein or associated therewith.

In any and/or all of the embodiments described herein, the apparatus 100, and/or any of the herein-described central processing computer(s) 10, the provider computer(s) 20, the payer computers(s) 30, the patient computer(s) 40, and the intermediary computer(s) 50, or any one or more of the pacemakers 60, the pacemaker monitors 65, the defibrillators 70, or the defibrillator monitors 75, can be programmed to perform troubleshooting, diagnostics, and repair, and/or reprogramming, of any of the herein-described pacemakers 60, pacemaker monitors 65, defibrillators 70, or defibrillator monitors 75.

In any and/or all of the embodiments described herein, the apparatus 100, and/or any of the herein-described central processing computer(s) 10, the provider computer(s) 20, the payer computers(s) 30, the patient computer(s) 40, and the intermediary computer(s) 50, or any one or more of the pacemakers 60, pacemaker monitors 65, defibrillators 70, or defibrillator monitors 75, can be utilized to update any setting(s) of any of the herein-described pacemakers 60 or defibrillators 70 in order to illicit a desired response in the individual or patient or from the individual or patient.

In any and/or all of the embodiments described herein, the apparatus 100, and/or any of the herein-described central processing computer(s) 10, the provider computer(s) 20, the payer computers(s) 30, the patient computer(s) 40, and the intermediary computer(s) 50, or any one or more of the pacemakers 60, pacemaker monitors 65, defibrillators 70, or defibrillator monitors 75, can be programmed to send an emergency telephone call to a 911 facility, to a police department, to afire department, to an emergency medical services department, or any other entity in order to report an emergency, a life-threatening condition, or any other emergency situation.

In any and/or all of the embodiments described herein, any of the pacemaker monitors 65 or defibrillator monitors 75 can be provided as a wearable device, as a bedside device, as a stand alone device, as a portable device, as a device which can be located in, or integrated in or with a vehicle, such as a motor vehicle, an automobile, a mass transportation vehicle, a bus, a train, a subway, a boat or marine vessel or vehicle, an aircraft, an airplane, a jet, a helicopter, and/or an ambulance or emergency vehicle or any land, air, or sea, variety, kind, or type. In such an embodiment, the respective pacemaker monitor 65 or defibrillator monitor 75 can be programmed to automatically detect the presence of a pacemaker 60 or a defibrillator 70, its model, type, or kind, and can be programmed to become automatically activated, or to switch on automatically, in order to monitor and/or service the pacemaker 60 or the defibrillator 70.

In any and/or all of the embodiments described herein, any of the pacemaker monitors 65 or defibrillator monitors 75 can be integrated with a respective pacemaker 60 or defibrillator 70.

In another preferred embodiment, the respective pacemaker monitor 65 and/or defibrillator monitor 75 can be programmable in order to allow any person or caregiver to be able to provide care for an individual or patient. In this regard, any problems arising relating to a respective pacemaker 60 or defibrillator 70 can be dealt with more rapidly.

In any and/or all of the embodiments described herein, a respective pacemaker monitor 65 or defibrillator monitor 75 can be a stand alone device which can be equipped and/or programmed so as to perform any and/or all functions of a programmer device and/or any of the herein-described functions or functionality described herein as being performed by the apparatus 100 or any of the provider computer(s) 20, payer computers(s) 30, patient computer(s) 40, or intermediary computer(s) 50 described herein.

In another preferred embodiment, a respective pacemaker monitor 65 or defibrillator monitor 75 can also contain a database which can store any of the herein-described healthcare records, files, or histories, or medical records, files, of histories, of or for the individual or patient. In another preferred embodiment, a respective pacemaker monitor 65 or defibrillator monitor 75 can also be equipped and/or programmed to make 911 emergency calls, collect symptom data and/or information from the individual or patient, perform a systematic check of the respective pacemaker 60 or defibrillator, and/or perform a systematic self-check or self-test.

In another preferred embodiment, a respective pacemaker monitor 65 or defibrillator monitor 75 can also be equipped and/or programmed to provide a voice synthesizer which can ask questions of the individual or patient, record the questions and answer to same, transmit the questions and answers, in dialogue form, to any one of more of the central processing computer(s) 10, the provider computer(s) 20, the payer computers(s) 30, the patient computer(s) 40, and the intermediary computer(s) 50, for storage in the respective healthcare records, files, or histories, or medical records, files, of histories, of or for the individual or patient, and/or transmit the questions and answers, in dialogue form, in a 911 emergency call, or to a communication device in a call to a provider, a hospital, an emergency medical technician, or any other emergency worker or healthcare worker.

In any and/or all of he embodiments described herein, any of the herein-described central processing computer(s) 10, provider computer(s) 20, payer computers(s) 30, patient computer(s) 40, and/or intermediary computer(s) 50, or any one or more of the pacemakers 60, pacemaker monitors 65, defibrillators 70, or defibrillator monitors 75, can be voice-activated and/or voice-controllable.

In any and/or all of the embodiments described herein, the patient computer 40 can be any computer, personal computer, cellular telephone, a personal digital assistant, a smartphone, or a tablet, or any other devices described herein as being used as a patient computer 40. In another preferred embodiment, a respective patient computer 40 can collect, store, and utilize, voice commands. In another preferred embodiment, a respective patient computer 40 can also be programmed to generate messages, e-mail messages, test messages, SMS messages, or any other message and can transmit same to any of the herein-described central processing computer(s) 10, provider computer(s) 20, payer computers(s) 30, patient computer(s) 40, and/or intermediary computer(s) 50, for storage in a respective healthcare record, file, or history, or medical record, file, of history, of or for the individual or patient, and/or can transmit same to any appropriate or respective computer or communication device used by or associated with a provider, a payer, a caregiver, a family member, a next of kin, or any other authorized or designated person or entity. In another preferred embodiment, any message(s) generated by and transmitted by the patient computer 40 can also be or can include a transcript of what the individual or patient says, a translation from one language to another language of what the individual or patient says, or can be or can include a message which is typed by the individual or patient or by another person.

In any and/or all of the embodiments described herein, the apparatus 100 or the central processing computer 10 can provide for any scheduled, random, on-the-fly, one-time, or any other, software and/or firmware upgrades, changes, settings, or calibrations, to and for any of the herein-described provider computer(s) 20, payer computers(s) 30, patient computer(s) 40, and/or intermediary computer(s) 50, or to and for any one or more of the pacemakers 60, pacemaker monitors 65, defibrillators 70, or defibrillator monitors 75, with a record of each upgrade to each respective computer or device being stored in the database 10H of the central processing computer 10 as well as in each respective computer or device.

In any and/or all of the embodiments, described herein, any and/or all of the hardware, software, and/or functionality described herein as being performed by any of the herein-described central processing computer(s) 10, the provider computer(s) 20, the payer computers(s) 30, the patient computer(s) 40, and the intermediary computer(s) 50, or any one or more of the pacemakers 60, the pacemaker monitors 65, the defibrillators 70, or the defibrillator monitors 75, as well as any of the hardware, software, and/or functionality of any of the herein-described central processing computer(s) 10, the provider computer(s) 20, the payer computers(s) 30, the patient computer(s) 40, and the intermediary computer(s) 50, or any one or more of the pacemaker monitors 65 or the defibrillator monitors 75, can be integrated designed into, built into, or integrated with any cellular telephone, smartphone, personal digital assistant, personal computer, laptop computer, notebook computer, tablet, or any other personal electronic device, used by or associated with any of the herein-described providers, payers, intermediaries, third parties, caregivers, relatives, any other authorized individuals or entities, or any individuals or patients themselves.

In another preferred embodiment, any patient computer or patient communication device 40 can serve as a respective pacemaker monitor 65, defibrillator monitor 75, or programmer device, and can perform any of the functions described herein as being performed by a respective pacemaker monitor 65, defibrillator monitor 75, or programmer device, or which are known, by those having skill in the art, can be performed by a respective pacemaker monitor 65, defibrillator monitor 75, or programmer device.

In a preferred embodiment, the apparatus 100 of the present invention can be utilized on or over, or in connection with, the Internet and/or the World Wide Web and/or on or over, or in connection with, any wireless communication networks or systems.

In any and/or all of the embodiments described herein, the apparatus 100 can allow any provider, payer, intermediary, or the individual or patient, or any other person, to access and/or link via a healthcare record, file, or history, or a medical record, file, or history, to a pacemaker 60, pacemaker monitor 65, defibrillator 70, or defibrillator monitor 75, and to perform any of the herein-described, or any other action, function, and operation. In another preferred embodiment, the apparatus 100 can also allow a respective provider, intermediary, caregiver, or any other person or entity, to submit a claim for payment to an insurer or payer for any action, function, operation, or service, performed.

In any and/or all of the embodiments described herein, any provider, payer, intermediary, or the individual or patient, or any other person, can perform spot checks or periodic checks on the operation of any of the herein-described pacemakers 60, pacemaker monitors 65, defibrillators 70, or defibrillator monitors 75.

In any and/or all of the embodiments described herein, any and/or all of the activities, events, occurrences, or happenings, concerning or regarding a respective pacemaker 60, pacemaker monitor 65, defibrillator 70, or defibrillator monitor 75, or any individual or patient using same, can be recorded and stored by the apparatus 100 and/or by any of the central processing computer(s) 10, provider computer(s) 20, payer computers(s) 30, patient computer(s) 40, or intermediary computer(s) 50 described herein.

The apparatus 100 and method of the present invention can provide an apparatus and a method which can perform any of the functions or functionality described herein as being capable of being provided by the apparatus 100 and method of the present invention.

While the present invention has been described and illustrated in various preferred and alternate embodiments, such descriptions are merely illustrative of the present invention and are not to be construed to be limitations thereof. In this regard, the present invention encompasses all modifications, variations and/or alternate embodiments, with the scope of the present invention being limited only by the claims which follow.

What is claimed is:

1. A computer-implemented method, comprising:
accessing, with a communication device, a computer in which is stored a healthcare record, file, or history, or a medical record, file, or history, of, for, or regarding, an individual or a patient;
accessing, with the communication device, the healthcare record, file, or history, or the medical record, file, or history, of, for, or regarding, the individual or the patient;
accessing or linking to a pacemaker, a pacemaker monitor, a defibrillator, or a defibrillator monitor, of or associated with the individual or the patient, with or using the communication device; and
performing, via, with, or using, the communication device, a control, a monitoring, a diagnostic, a programming, a re-programming, a repair, program, a calibration, a setting, or a re-setting, action, operation, or function, on, regarding, or relating to, the pacemaker, the pacemaker monitor, the defibrillator, or the defibrillator monitor.

2. The computer-implemented method of claim 1, further comprising:
storing, in the healthcare record, file, or history, or in the medical record, file, or history, of, for, or regarding, the individual or the patient, data or information regarding the action, operation, or function, performed on, regarding, or relating to, the pacemaker, the pacemaker monitor, the defibrillator, or the defibrillator monitor.

3. The computer-implemented method of claim 2, further comprising:
generating, with the computer, an insurance claim or an insurance claim form which is suitable for being transmitted to or submitted to an insurer or a payer of or for the individual or the patient.

4. The computer-implemented method of claim 3, further comprising:
transmitting, from a transmitter or from the computer, the insurance claim or the insurance claim form to a second computer or to a second communication device, wherein the second computer or the second communication device is associated with an insurer or a payer of or for the individual or the patient.

5. The computer-implemented method of claim 4, further comprising:
storing information regarding the insurance claim or the insurance claim form in the computer, or in the healthcare record, file, or history, or in the medical record, file, or history, of, for, or regarding, the individual or the patient, after the insurance claim or the insurance claim form has been transmitted to the second computer or to the second communication device.

6. The computer-implemented method of claim 1, further comprising:
receiving, with the computer or with a second communication device, a notification message or an alert which is transmitted from the pacemaker, the pacemaker monitor, the defibrillator, or the defibrillator monitor.

7. The computer-implemented method of claim 6, further comprising:
storing information regarding notification message or the alert in the computer, or in the healthcare record, file, or history, or in the medical record, file, or history, of, for, or regarding, the individual or the patient.

8. The computer-implemented method of claim 1, further comprising:
uploading, from the computer or from an external device or system, a software update or a firmware update to the pacemaker, the pacemaker monitor, the defibrillator, or the defibrillator monitor.

9. The computer-implemented method of claim 1, wherein the communication device is associated with or used by a provider or a healthcare provider of the individual or the patient.

10. The computer-implemented method of claim 1, wherein the communication device is associated with or used by a payer or an insurer of the individual or the patient.

11. The computer-implemented method of claim 1, wherein the communication device is associated with or used by the individual or the patient.

12. The computer-implemented method of claim 1, wherein the communication device is associated with or used by a friend, a relative, or a caregiver, of the individual or the patient.

13. The computer-implemented method of claim 1, wherein the communication device is associated with or used by a manufacturer, a seller, or a service representative or entity, of or for the pacemaker, the pacemaker monitor, the defibrillator, or the defibrillator monitor.

14. The computer-implemented method of claim 1, wherein the pacemaker, the pacemaker monitor, the defibrillator, or the defibrillator monitor, is a pacemaker.

15. The computer-implemented method of claim 1, wherein the pacemaker, the pacemaker monitor, the defibrillator, or the defibrillator monitor, is a pacemaker monitor.

16. The computer-implemented method of claim 1, wherein the pacemaker, the pacemaker monitor, the defibrillator, or the defibrillator monitor, is a defibrillator.

17. The computer-implemented method of claim 1, wherein the pacemaker, the pacemaker monitor, the defibrillator, or the defibrillator monitor, is a defibrillator monitor.

18. The computer-implemented method of claim 1, wherein the communication device is a computer or a personal computer.

19. The computer-implemented method of claim 1, wherein the communication device is a cellular telephone, a personal digital assistant, or a tablet.

20. The computer-implemented method of claim 1, wherein the communication device is an interactive television.
